(19) United States
(12) Patent Application Publication
(10) Pub. No.: US 2026/0345826 A1
(43) Pub. Date: Nov. 5, 2026

(54) COMPOSITIONS AND METHODS FOR TREATING WILSON'S DISEASE

(71) Applicant: HYDROGENE THERAPEUTICS, INC., Houston, TX (US)

(72) Inventor: Robert Kruse, Houston, TX (US)

(73) Assignee: HYDROGENE THERAPEUTICS, INC., Houston, TX (US)

(21) Appl. No.: 19/107,568

(22) PCT Filed: Nov. 21, 2023
(86) PCT No.: PCT/US2023/031730

§ 371 Date: Feb. 27, 2025

(51) Int. Cl. A61K 48/00

(52) U.S. Cl. CPC A61K 48/0058; A61K 48/0075

(57) ABSTRACT

Methods and composition of treating Wilson's Disease with a non-viral, DNA vector is provided. The composition consists of descriptions of an ATP7B gene driven by expression elements that yield high-level expression. Delivery of this composition successful yields a proliferative advantage for liver cells harboring the vector in Wilson's Disease liver, such that those cells will increase in number over time. The DNA vector also harbors unique sequence variants of ATP7B with increased activity for gene therapy. Vector designs that allow for mitotic replication are also provided. Methods of administering the DNA vector into the liver through naked delivery of hydrodynamic injection are disclosed.

### CROSS-REFERENCE TO RELATED APPLICATIONS

The present application is related to and claims priority under 35 U.S.C. § 119(e) to U.S. Provisional Patent Application No. 63/374,229, entitled “COMPOSITIONS AND METHODS FOR TREATING WILSON'S DISEASE,” filed Aug. 31, 2022. The entire contents of the aforementioned patent application is incorporated herein by this reference.

### FIELD

The present disclosure relates to compositions and methods for treating Wilson's disease. More particularly, the present disclosure relates to compositions and methods for treating Wilson's disease by gene therapy administered via hydrodynamic injection.

### BACKGROUND

Wilson's disease is a genetic disorder associated with mutation of the ATP7B gene, which disrupts copper metabolism such that excess copper builds up in the afflicted individual's body. Normally, copper is absorbed in the small intestine via enterocyte uptake by hCTR1, and copper transport into the blood is mediated by ATP7B at the basolateral aspect of duodenal epithelia. Copper is then conveyed by portal circulation to the liver, where excess copper is removed by excretion into the bile at the apical aspect of hepatocytes, a process that is disrupted by mutations in the ATP7B gene.

Phenotypically, Wilson's disease generally presents with neurological and/or liver-related symptoms, and while symptoms usually begin between the ages of 5 and 35 years, both early onset (e.g., infancy) and late onset (e.g., >70 years old) presentations have been documented. Diagnosis may be difficult and often involves a combination of blood tests, urine tests and/or liver biopsy. Neurological symptoms may include tremors, muscle stiffness, trouble in speaking, personality changes, anxiety, and psychosis, while liver-related symptoms may include vomiting, weakness, fluid buildup in the abdomen, swelling of the legs, yellowish skin and itchiness. Complications of Wilson's disease may include increased chances of liver failure, liver cancer, and/or kidney damage.

Wilson's disease is a monogenic, autosomal recessively inherited condition associated with mutation of a copper-transporting P-type ATPase (i.e., ATP7B). For a person to be affected, they must inherit a mutated copy of the gene from both parents. More than 500 ATP7B mutations have now been identified, including missense mutations, small deletions/insertions in the coding region, or splice junction mutations. Genetic testing may be used to screen family members of those affected. Early estimates suggested that Wilson's disease occurs at a frequency of about 1 in 30,000 people; however, more recently it has been suggested that the frequency is much higher (e.g., 1 in 7,000 people).

Wilson's disease is typically treated with dietary changes (e.g., a low copper diet) and medication (e.g., chelating agents such as trientine and d-penicillamine, and also zinc supplements). Unfortunately, the metabolic nature of Wilson's disease necessitates that medical therapy is a lifelong protocol because copper accumulation in the afflicted individual cannot be completely controlled by a low copper diet. Accordingly, there is an urgent need for medical therapies that can treat and/or prevent this debilitating disease.

### SUMMARY

The present disclosure relates to compositions and methods for treating Wilson's disease. More particularly, the present disclosure relates to compositions and methods for treating Wilson's disease by gene therapy. As described in detail below, the present disclosure is based, at least in part, on the surprising discovery that non-viral modification of hepatocytes with ATP7B gene in Wilson's Disease (WD) liver can lead to expansion of those hepatocytes through a proliferative advantage.

In an aspect, the present disclosure provides a method of treating of Wilson's Disease, including the steps of: a) administering a non-viral DNA vector capable stably expressing human ATP7B into liver cells by hydrodynamic injection, and b) yielding a selective proliferative advantage of liver cells harboring the DNA vector in a Wilson's Disease individuals' liver over time increasing the amount of those liver cells, wherein no other exogenous agents or chemicals or partial hepatectomy are needed to induce this proliferative advantage.

In exemplary embodiments, the liver cells are hepatocytes and cholangiocytes.

In exemplary embodiments, the non-viral DNA vector is integrated into host hepatocytes or cholangiocytes with the use of a transposon system for replication with mitosis.

In exemplary embodiments, the transposon system is a piggyBac transposon or Sleeping Beauty Transposon.

In exemplary embodiments, non-viral DNA vector alternatively harbors sequence elements to enable episomal replication for replication with mitosis.

In exemplary embodiments, the episomal replication sequence is a scaffold/matrix attachment region sequence.

In exemplary embodiments, the administered vector is double-stranded circular or linear DNA.

In exemplary embodiments, non-viral DNA vector is at least a promoter, a 5′ UTR, a human ATP7B coding sequence, a 3′ UTR, an enhancer and polyadenylation sequence.

In exemplary embodiments, the promoter is selected from the group consisting of a hepatocyte-specific promoter, consisting of alpha-1 antitrypsin, human thyroxine binding globulin, hemopexin, albumin, and HBV core promoters.

In exemplary embodiments, the promoter is selected from the group consisting of a cholangiocyte-specific promoter, consisting of cytokeratin-17, cytokeratin-19, cyclooxygenase-2 (COX-2), midkine (MK), mucin-1 (MUC1), and osteopontin.

In exemplary embodiments, the promoter is a tandem of a hepatocyte- and cholangiocyte-specific promoter, thereby allowing ATP7B expression in both hepatocytes and cholangiocytes.

In exemplary embodiments, a promoter is selected that has expression in both hepatocytes and cholangiocytes, such as cytokeratin-18 promoter.

In exemplary embodiments, the enhancer element is added to the promoter, consisting of a liver-specific enhancer such as human apolipoprotein hepatic control region, human albumin enhancer, human ApoE enhancer, or a viral enhancer such as SV40 enhancer, HBV enhancer I, HBV enhancer II to drive more potent expression.

In exemplary embodiments, non-viral DNA vector optionally contains at least one intron selected from SV40 intron, Minute Virus of Mice (MVM) intron, and human growth hormone (HGH) intron, preferably in the 5′ UTR to enhance ATP7B expression.

In exemplary embodiments, the coding sequence for ATP7B is codon-optimized for human hepatocyte expression.

In exemplary embodiments, the 3′ UTR is selected among human beta-globin UTR, human alpha-globin UTR, or albumin UTR.

In exemplary embodiments, the non-viral DNA vector does not induce overexpression toxicity of ATP7B in large animals compared to rodents when utilizing constitutively active liver-specific promoters In exemplary embodiments, hydrodynamic injection of plasmid DNA occurs through the biliary tract, hepatic vein, or hepatic artery to mediate delivery into liver cells.

In exemplary embodiments, hepatocytes expressing ATP7B after non-viral delivery will expand at least 2-fold, 3-fold, 4-fold, or 5-fold or more after at least 2 months.

In exemplary embodiments, wherein the ATP7B protein coding sequence can tolerate C-terminal additions for protein tracking without affecting protein function.

In one aspect, the disclosure provide a method of treating a human subject having Wilson's Disease, comprising administering a non-viral DNA vector into liver cells by hydrodynamic injection, wherein a therapeutically effective dose of the non-viral DNA vector is administered to the human subject via the biliary system into a liver of the human subject.

In exemplary embodiments, the administration step occurs via an endoscopic retrograde cholangio-pancreatography procedure.

In exemplary embodiments, the non-viral DNA vector achieves expression in at least 20% of hepatocytes in the human subject after delivery, as detectable by protein or RNA staining.

In exemplary embodiments, the non-viral DNA vector dose is at least about 1 mg, 2 mg, 3 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg of DNA per kilogram of liver weight of the human subject.

In exemplary embodiments, the subject exhibits normalization of their liver copper content and restoration of ceruloplasmin levels.

In exemplary embodiments, the human subject is administered with a copper chelation or zinc therapy prior to administration of the non-viral DNA vector.

In exemplary embodiments, the hepatic pathology of the human subject is normalized and disruption of the efficiency of biliary hydrodynamic gene delivery is avoided.

In exemplary embodiments, the copper chelation therapy is selected for D-penicillamine (DPA) or trientine (TETA).

In exemplary embodiments, the human subject possesses normal ALT and AST liver enzymes.

In exemplary embodiments, the human subject possesses liver enzyme elevations within 3 times the upper limit of normal, to assume close to normal liver histology.

In exemplary embodiments, the human subject have elevated liver enzymes and still require treatment, then the human subject requires a DNA dose at least 50 mg per kilogram of liver weight to compensate for distorted pathology.

In exemplary embodiments, the human subject has fulminant hepatitis induced by Wilson's Disease and is administered via an endoscopic procedure with an elevated non-viral DNA dose of at least 50 mg per kg of liver weight to compensate for distorted pathology.

In exemplary embodiments, the efficacy of the non-viral DNA vector is monitored with urinary copper, serum copper, ceruloplasmin, and/or biopsy-gained hepatic copper measurements.

In exemplary embodiments, the subject is preferentially selected among patients with Wilson's disease who do not tolerate current copper chelation medications, or alternatively have neurological disease with any treatment response, or alternatively have antibodies against AAV capsids.

In exemplary embodiments, after the administration step, the patient may start or continue taking copper chelation medications and zinc for a period of at least 1-month, at least 2 months, at least 3 months, or at least 6 months to help accelerate the de-coppering process from the body, before cessation of pharmacologic therapy.

In exemplary embodiments, the combination therapy will prevent hepatocyte turnover from copper-induced death before the genetic treatment has time to take effect.

In exemplary embodiments, the non-viral DNA vector is maintained either as an episome without integration, or alternatively is facilitated with integration.

In exemplary embodiments, the human subject is redosed with episomal vectors configured for redosing at least once every one year, at least every two years, at least every three years, or at least every five years.

In exemplary embodiments, the need for redosing is determined by assessing the elevation in the ALT and AST liver enzymes, such that enzymes fall outside the upper limit of normal.

In exemplary embodiments, the non-viral DNA is integrated in the genome of the human subject, optionally via a transposase, a large serine recombinase, or a CRISPR-directed homologous recombination.

In exemplary embodiments, the non-viral DNA vector yields a selective proliferative advantage of liver cells harboring the DNA vector in a Wilson's Disease individuals' liver over time increasing the amount of those liver cells positive for the vector-derived ATP7B, optionally wherein no other exogenous agents or chemicals or partial hepatectomy are needed to induce this proliferative advantage, optionally wherein the proliferative advantage can be slowed through the administration of copper chelation and zinc as desired.

In exemplary embodiments, hepatocytes and cholangiocytes of the liver are targeted for expression with ATP7B.

In exemplary embodiments, the non-viral DNA vector is integrated into host hepatocytes or cholangiocytes for replication with mitosis to provide stability due to the proliferative advantage and/or turnover of un-transfected hepatocytes, optionally via a transposon system.

In exemplary embodiments, the transposon system is a piggyBac transposon, a hyperactive piggyBac transposon, or a Sleeping Beauty Transposon.

In exemplary embodiments, the non-viral DNA vector alternatively harbors sequence elements to enable episomal replication for replication with mitosis.

In exemplary embodiments, the episomal replication sequence is a scaffold/matrix attachment region sequence.

In exemplary embodiments, the administered non-viral vector is a double-stranded circular or linear DNA.

In exemplary embodiments, the non-viral DNA vector includes a promoter, a 5′ UTR, a human ATP7B coding sequence, a 3′ UTR, an enhancer and polyadenylation sequence.

In exemplary embodiments, the promoter is selected from the group consisting of a hepatocyte-specific promoter, consisting of alpha-1 antitrypsin, human thyroxine binding globulin, hemopexin, albumin, LP1, P3, and mouse transthyretin promoters.

In exemplary embodiments, the promoter is selected from the group consisting of a cholangiocyte-specific promoter, consisting of cytokeratin-17, cytokeratin-19, cyclooxygenase-2 (COX-2), midkine (MK), mucin-1 (MUC1), and osteopontin.

In exemplary embodiments, the promoter is a tandem of a hepatocyte- and cholangiocyte-specific promoter, with the cholangiocyte 5′ in order to the hepatocyte promoter, thereby allowing ATP7B expression in both hepatocytes and cholangiocytes.

In exemplary embodiments, a promoter is selected that has expression in both hepatocytes and cholangiocytes, such as cytokeratin-18 promoter, or the alpha-1 antitrypsin promoter.

In exemplary embodiments, the enhancer element is added to the promoter, consisting of a liver-specific enhancer such as human apolipoprotein hepatic control region, human albumin enhancer, human ApoE enhancer, or a viral enhancer such as HBV enhancer I, HBV enhancer II to drive more potent expression.

In exemplary embodiments, non-viral DNA vector optionally contains at least one intron selected from SV40 intron, Minute Virus of Mice (MVM) intron, and human growth hormone (HGH) intron, preferably in the 5′ UTR to enhance ATP7B expression.

In exemplary embodiments, the coding sequence for ATP7B is codon-optimized for human hepatocyte expression, such that the expression level is at least 2-fold higher than the wild-type ATP7B sequence.

In exemplary embodiments, the 3′ UTR is selected among human beta-globin UTR, human alpha-globin UTR, or a doublet of those sequences for added stability.

In exemplary embodiments, the non-viral DNA vector does not induce overexpression toxicity of ATP7B in large animals compared to rodents when utilizing constitutively active liver-specific promoters due to differences in delivery efficiency, such that no native regulatory elements are required.

In exemplary embodiments, expression of ATP7B is constitutive and thus can suppress the endogenous mutant ATP7B, which is regulated by copper levels, thereby enhancing the therapeutic effect by alleviating the malfunctioning form inside the cell.

In exemplary embodiments, wherein the native ATP7B promoter, or other promoter controlled by metal-responsive elements are preferably not used in the viral vector to avoid promoter competition with the endogenous mutant proteins.

In exemplary embodiments, the subject is further administered with copper supplements if vector expression from ATP7B be too significant and cause copper deficiency In exemplary embodiments, the native ATP7B mutant's expression is knocked down with shRNA that is also encoded on the non-viral vector, such that the new delivered wildtype ATP7B avoids mispairing with the mutant receptor inside the cell.

In exemplary embodiments, hydrodynamic injection of DNA occurs alternatively through the hepatic vein, or hepatic artery to mediate delivery into liver cells.

In exemplary embodiments, hepatocytes expressing ATP7B after non-viral delivery will expand at least 2-fold, 3-fold, 4-fold, or 5-fold or more after at least 2 months post-gene delivery compared to their original number post-injection.

In exemplary embodiments, the ATP7B protein coding sequence can tolerate an N-terminal or C-terminal additions for protein identification without affecting protein function.

In exemplary embodiments, the subject is a canine subject who possesses a pathologic mutation in ATP7B and elevated hepatic copper levels causing silent or active hepatitis.

In exemplary embodiments, the canine subject will receive wildtype canine ATP7B (NM_001025267.1) into the canine liver.

In exemplary embodiments, the canine subject will preferentially receive integrative vector strategies to avoid the need for future redosing.

### DEFINITIONS

To facilitate an understanding of the present disclosure, a number of terms and phrases are defined below:

Unless specifically stated or obvious from context, as used herein, the term “about” is understood as being within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

By “agent” is meant any small molecule chemical compound, antibody, nucleic acid molecule, polypeptide, or fragments thereof.

By “ameliorate” is meant decrease, suppress, attenuate, diminish, arrest, or stabilize the development or progression of a disease (e.g., a Wilson's disease phenotype).

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical but has analogous functional or structural features. For example, an ATP7B analog retains the biological activity of a corresponding naturally occurring ATPase, while having certain biochemical modifications that enhance the analog's function relative to a naturally occurring polypeptide. Such biochemical modifications could increase the analog's protease resistance or half-life, without altering, for example, copper transport. An analog may include an unnatural or synthetic amino acid or altered amino acid sequences within the ATP7B protein.

The phrase “combination therapy” embraces the administration of a gene therapy protocol and one or more additional therapeutic agents (e.g., copper chelating compounds) as part of a specific treatment regimen intended to provide a beneficial (additive or synergistic) effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days, or weeks depending upon the combination selected). “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous or overlapping manner. Substantially simultaneous administration can be accomplished, for example, by administering to the subject, for example, one or more copper chelating compounds while administering a gene therapy protocol as disclosed herein. Sequential or substantially simultaneous administration of each therapeutic agent can be affected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection (e.g., a gene therapy protocol) while the other component(s) (e.g., one or more copper chelating compounds) of the combination may be administered orally. The components may be administered in any therapeutically effective sequence.

The phrase “combination” embraces groups of compounds and/or non-drug gene therapies useful as part of a combination therapy as disclosed herein.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “control” is meant a standard or reference condition.

By “disease” is meant any condition or disorder (e.g., Wilson's disease) that damages or interferes with the normal function of a cell, tissue, or organ.

By “effective amount” is meant the amount required to ameliorate the symptoms of a disease (e.g., neurological or liver symptoms of Wilson's disease) relative to an untreated patient. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. The effective amount may also refer to levels of gene expression (e.g., ATP7B mRNA or protein expression) in the appropriate tissues of a patient.

By “fragment” is meant a portion of a polypeptide or nucleic acid molecule. This portion contains, preferably, at least 5%, 10%, 20%, 30%, 40%, 50%, 60%, 70%, 80%, or 90% of the entire length of the reference nucleic acid molecule or polypeptide. A fragment may contain 5, 10, 20, 30, 40, 50, 60, 70, 80, 90, or 100, 200, 300, 400, 500, 600, 700, 800, 900, 1000, 2000, 3000, 4000, 5000 or more nucleotides or amino acids.

A “gene therapy composition” is to be understood as meaning a DNA composition (e.g., including a full length ATP7B nucleotide sequence, or portion thereof) for generating prophylaxis and/or treatment of Wilson's disease. Accordingly, gene therapy compositions are medicaments which comprise a full length ATP7B nucleotide sequence, or portion thereof, and are intended to be used in humans or animals for generating prophylaxis and/or treatment of Wilson's disease.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “isolated polynucleotide” is meant a nucleic acid molecule (e.g., a DNA, an mRNA, a cDNA, and the like) that is free of the genes from which, in the naturally occurring genome of the organism, the nucleic acid molecule of the disclosure is normally associate or derived. The term therefore includes, for example, a recombinant DNA (e.g., including a genomic DNA or cDNA coding for a ATP7B gene, as well as associated regulatory components such as, for example, an enhancer(s), a promoter, 5′ and/or 3′ untranslated regions (UTRs), and the like) that may be incorporated into: a vector, or an autonomously replicating plasmid or virus, or into the genomic DNA of a prokaryote or eukaryote, or into a polynucleotide that exists as a separate molecule (for example, a cDNA or a genomic or cDNA fragment produced by PCR or restriction endonuclease digestion, or a naked DNA construct such as a plasmid or cosmid or linear DNA) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

“Mutation” for the purposes of this disclosure means a DNA sequence found in the ATP7B gene of a patient that does not correlate with an established wildtype ATP7B gene sequence, and such mutations may be due to one or more single nucleotide polymorphisms, one or more deletions or insertions of one or more nucleotides, and deletion or insertion of splice site junctions. “Mutation” may also refer to patterns in the sequence of RNA from a patient that are not attributable to expected variations based on known information for the ATP7B gene and are reasonably considered to be novel variations in, for example, the splicing pattern of the ATP7B gene of the patient.

Unless specifically stated or obvious from context, as used herein, the term “or” is understood to be inclusive. Unless specifically stated or obvious from context, as used herein, the terms “a,” “an,” and “the” are understood to be singular or plural.

The term “patient” or “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

“Pharmaceutically acceptable” refers to approved or approvable by a regulatory agency of the Federal or a state government or listed in the U.S. Pharmacopeia or other generally recognized pharmacopeia for use in animals, including humans.

“Pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of pooled tumor specific neo-antigens as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize further pharmaceutically acceptable salts for the pooled tumor specific neo-antigens provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, refer to reducing the probability of developing a disease or condition (e.g., Wilson's disease) in a subject, who does not have, but is at risk of or susceptible to developing the disease or condition (e.g., Wilson's disease).

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

By “reduces” is meant a negative alteration of at least 10%, 25%, 50%, 75%, or 100%.

By “reference” is meant a standard or control condition.

A “reference sequence” is a defined sequence used as a basis for sequence comparison (e.g., a wildtype ATP7B gene sequence; Genebank sequence NM_000053). A reference sequence may be a subset of, or the entirety of, a specified sequence; for example, a segment of a full-length cDNA or genomic sequence, or the complete cDNA or genomic sequence. For polypeptides, the length of the reference polypeptide sequence will generally be at least about 10-5,000 amino acids, 10-4,000 amino acids, 10-3,000 amino acids, 10-2,000 amino acids, 10-1,500 amino acids, 10-1,000 amino acids, 10-500 amino acids, or 10-100 amino acids. Preferably, the length of the reference polypeptide sequence may be at least about 10-50 amino acids, more preferably at least about 10-40 amino acids, and even more preferably about 10-30 amino acids, about 10-20 amino acids, about 15-25 amino acids, or about 20 amino acids. For nucleic acids, the length of the reference nucleic acid sequence will generally be at least about 50 nucleotides, about 60 nucleotides, about 75 nucleotides, about 100 nucleotides, about 200 nucleotides, about 300 nucleotides, about 400 nucleotides, about 500 nucleotides, about 750 nucleotides, about 1000 nucleotides, about 1250 nucleotides, about 1500 nucleotides, about 1750 nucleotides, about 2000 nucleotides, about 2250 nucleotides, about 2500 nucleotides, about 2750 nucleotides, about 3000 nucleotides, about 3250 nucleotides, about 3500 nucleotides, about 3750 nucleotides, about 4000 nucleotides, about 4250 nucleotides, about 4500 nucleotides, about 4750 nucleotides, about 5000 nucleotides, about 5250 nucleotides, about 5500 nucleotides, about 5750 nucleotides, about 6000 nucleotides, about 6250 nucleotides, about 6500 nucleotides, about 6750 nucleotides, about 7000 nucleotides, about 7250 nucleotides, about 7500 nucleotides, about 7750 nucleotides, or about 8000 nucleotides or any integer thereabout or there between.

Nucleic acid molecules useful in the methods of the disclosure include any nucleic acid molecule that encodes a polypeptide (e.g., an ATP7B polypeptide) of the disclosure, or a fragment thereof. Such nucleic acid molecules need not be 100% identical with an endogenous nucleic acid sequence, but will typically exhibit substantial identity (e.g., 100%, 99%, 98%, 97%, 96%, 95%, 94%, 93%, 92%, 91%, or 90%). Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., an ATP7B gene described herein), or portions thereof, under various conditions of stringency. (see, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30 °C, more preferably of at least about 37 °C, and most preferably of at least about 42 °C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30 °C in 750 mM NaCl, 75 mM trisodium citrate, and 1% SDS. In a more preferred embodiment, hybridization will occur at 37 °C in 500 mM NaCl, 50 mM trisodium citrate, 1% SDS, 35% formamide, and 100 g/ml denatured salmon sperm DNA (ssDNA). In a most preferred embodiment, hybridization will occur at 42 °C in 250 mM NaCl, 25 mM trisodium citrate, 1% SDS, 50% formamide, and 200 g/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25 °C, more preferably of at least about 42 °C, and even more preferably of at least about 68 °C. In a preferred embodiment, wash steps will occur at 25 °C in 30 mM NaCl, 3 mM trisodium citrate, and 0.1% SDS. In a more preferred embodiment, wash steps will occur at 42 °C in 15 mM NaCl, 1.5 mM trisodium citrate, and 0.1% SDS. In a more preferred embodiment, wash steps will occur at 68 °C in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid or nucleotide sequence (for example, any one of the amino acid or nucleotide sequences described herein). Preferably, such a sequence is at least 60%, at least 70%, at least 80%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, or at least 100% identical at the amino acid sequence or nucleic acid sequence used for comparison (e.g., wildtype ATP7B).

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the terms “treat,” “treated,” “treating,” “treatment,” and the like refer to reducing or ameliorating a disorder and/or symptoms associated therewith (e.g., Wilson's disease). It will be appreciated that, although not precluded, treating a disorder or condition does not require that the disorder, condition, or symptoms associated therewith be completely eliminated.

The term “therapeutic effect” refers to some extent of relief of one or more of the symptoms (e.g., neurological, liver-related, etc.) of Wilson's disease or its associated pathology. “Therapeutically effective amount” as used herein refers to an amount of an agent or combination therapy which is effective, upon single or multiple dose administration to the cell or subject, in prolonging the survivability of the patient with Wilson's disease, reducing one or more signs or symptoms of Wilson's disease, preventing or delaying onset of symptoms of Wilson's disease, and the like, beyond what would be expected in the absence of such treatment. “Therapeutically effective amount” is intended to qualify the amount required to achieve a therapeutic effect. A physician or veterinarian having ordinary skill in the art can readily determine and prescribe the “therapeutically effective amount” agent or combination therapy required.

The pharmaceutical compositions typically should provide a dosage of from about 0.0001 mg to about 1500 mg of compound per day, preferably 50 mg to about 1500 mg of compound per day. For example, dosages for systemic administration to a human patient can range from about 0.01-10 μg/kg, about 20-80 μg/kg, about 5-50 μg/kg, about 75-150 μg/kg, about 100-500 μg/kg, about 250-750 μg/kg, about 500-1000 μg/kg, about 1-10 mg/kg, about 5-50 mg/kg, about 25-75 mg/kg, about 50-100 mg/kg, about 100-250 mg/kg, about 50-100 mg/kg, about 250-500 mg/kg, about 500-750 mg/kg, about 750-1000 mg/kg, about 1000-1500 mg/kg, about 1500-2000 mg/kg, about 5 mg/kg, about 20 mg/kg, about 50 mg/kg, about 75 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg, about 1000 mg/kg, about 1050 mg/kg, about 1100 mg/kg, about 1150 mg/kg, about 1200 mg/kg, about 1250 mg/kg, about 1300 mg/kg, about 1350 mg/kg, about 1400 mg/kg, about 1450 mg/kg, or about 1500 mg/kg. Pharmaceutical dosage unit forms are prepared to provide from about 50 mg to about 1500 mg, for example from about 100 to about 1000 mg of the compound or a combination of essential ingredients per dosage unit form.

By “Full-length Human ATP7B promoter nucleic acid molecule” is meant a polynucleotide encoding an ATP7B promoter. The following sequence starts at −895 before the transcription start site of human ATP7B and extends to +175 of transcription. This sequence is designed to stop after the final Sp1 binding site of the ATP7B promoter, containing a portion of the native ATP7B 5′ UTR. As used herein, a full-length human ATP7B promoter nucleic acid molecule has at least about 100% identity, 99% identity, 98% identity, 97% identity, 96% identity, 95% identity, 94% identity, 93% identity, 92% identity, 91% identity, or 90% identity to the following nucleic acid molecule sequence:

acaaggaaggccatttgcccgcaaaatttagctacactggacgggcaagtacccctacagaaga
gaaaacgtctgtgagcccacacgaccggctgctcacctcaacaacttgcacaggcaccagctcc
tttcgccggccgccatcttccgccgacccccgaactcaggaaacgcttcactttccttttccct
attggctcctgagaaagcaagccgtgctcgccccgcccccacgggccaattgtgcgttactatt
ggttactggtagccgcttcccacggccttccagccaatagaatatgccgaggcgtagactagtg
ttcggcgtggcgcacacggctcccgcccccgtgggcgggacagcagtggggggttgggctgagg
agggcgtggcctgtgattgacagccgtcgctccctccctcggccacctcccccactagaagccc
ccgcctgggcgcctgcgcccccgttcccggcccaaagcccgccgcccgttggaggccattggct
ggcctttgcgcacagcggatcgattttccaggtgcggagttcactcttgccgcggttgcttcct
ttgggacccacggcgtccggcagccaggcgcagagtccgaggagggggcagcgcagagcggacc
cgacgcggcgccgccgggcaccttccccgcaggcggtgggtgagccctgggagctgagtctgcg
gtccggctctgcgcagctcacctgccctcccgctcccgcacacgcgtgagatcccagtacagtg
tcggagcgcaccagcgcgaggtggccgagaccgcggaggaggacaggcctccgccctgcggcgc
cggcacggcagaggacattgtggcactggcacggcagagaacactgtggcaccggcggggccgg
cagttccagggtgggcactcccagccacctggggagtgggcgagggtccgaggcccactctccc
ctcacgctctcatccccgtgcccccaggtcgggaggacggcggcgcgcaactttgaatcatccg
tgtgaagagggctgcggcttccccggtcccaaatgaaggggcggtt

By “Truncated Human ATP7B promoter nucleic acid molecule” is meant a polynucleotide encoding a truncated ATP7B promoter without negative regulatory elements. This sequence deletes a metal response element-like sequence that is inhibitor to expression in the native context. As used herein, a truncated human ATP7B promoter nucleic acid molecule has at least about 100% identity, 99% identity, 98% identity, 97% identity, 96% identity, 95% identity, 94% identity, 93% identity, 92% identity, 91% identity, or 90% identity to the following nucleic acid molecule sequence:

cacgaccggctgctcacctcaacaacttgcacaggcaccagctcctttcgccggccgccatctt
ccgccgacccccgaactcaggaaacgcttcactttccttttccctattggctcctgagaaagca
agccgtgctcgccccgcccccacgggccaattgtgcgttactattggttactggtagccgcttc
ccacggccttccagccaatagaatatgccgaggcgtagactagtgttcggcgtggcgcacacgc
ctcccgcccccgtgggcgggacagcagtggggggttgggctgaggagggcgtggcctgtgattc
acagccgtcgctccctccctcggccacctcccccactagaagcccccgcctgggcgcctgcgcc
cccgttcccggcccaaagcccgccgcccgttggaggccattggctggcctttgcgcacagcgga
tcgattttccaggtgcggagttcactcttgccgcggttgcttcctttgggacccacggcgtccc
gcagccaggcgcagagtccgaggagggggcagcgcagagcggacccgacgcggcgccgccgggc
accttccccgcaggcggtgggtgagccctgggagctgagtctgcggtccggctctgcgcagctc
acctgccctcccgctcccgcacacgcgtgagatcccagtacagtgtcggagcgcaccagcgcga
ggtggccgagaccgcggaggaggacaggcctccgccctgcggcgccggcacggcagaggacatt
gtggcactggcacggcagagaacactgtggcaccggcggggccggcagttccagggtgggcact
cccagccacctggggagtgggcgagggtccgaggcccactctcccctcacgctctcatccccgt
gcccccaggtcgggaggacggcggcgcgcaactttgaatcatccgtgtgaagagggctgcggct
tccccggtcccaaatgaaggggcggtt

By “ATP7B promoter enhancer element nucleic acid molecule” is meant a polynucleotide encoding an ATP7B promoter enhancer element. As used herein, an ATP7B promoter enhancer element nucleic acid molecule has at least about 100% identity, 99% identity, 98% identity, 97% identity, 96% identity, 95% identity, 94% identity, 93% identity, 92% identity, 91% identity, or 90% identity to the following nucleic acid molecule sequence:

cacgaccggctgctcacctcaacaacttgcacaggcaccagctcctttcg
ccggccgccatcttccgccgacccccgaactcaggaaacgcttcactttc
cttttccctattggctcctgagaaagcaagccgtgctcgccccgccccca
cgggccaatt

By “SV40 intron sequence nucleic acid molecule” is meant a polynucleotide encoding an ATP7B promoter enhancer element. As used herein, a SV40 intron sequence nucleic acid molecule has at least about 100% identity, 99% identity, 98% identity, 97% identity, 96% identity, 95% identity, 94% identity, 93% identity, 92% identity, 91% identity, or 90% identity to the following nucleic acid molecule sequence:

CTCTAAGGTAAATATAAAATTTTTAAGTGTATAATGTGTTAAACTACTGA
TTCTAATTGTTTCTCTCTTTTAGATTCCAACCTTTGGAACTGA

By “ATP7B copper-transporting ATPase 2 isoform A polypeptide” is meant a polypeptide or fragment thereof having at least about 100% amino acid identity, 99% amino acid identity, 98% amino acid identity, 97% amino acid identity, 96% amino acid identity, 95% amino acid identity, 94% amino acid identity, 93% amino acid identity, 92% amino acid identity, 91% amino acid identity, or 90% amino acid identity to NCBI Reference Sequence: NP_000044.2, representing:

MPEQERQITAREGASRKILSKLSLPTRAWEPAMKKSFAFDNVGYEGGLDGLGPSSQVATSTVRI
LGMTCQSCVKSIEDRISNLKGIISMKVSLEQGSATVKYVPSVVCLQQVCHQIGDMGFEASIAEG
KAASWPSRSLPAQEAVVKLRVEGMTCQSCVSSIEGKVRKLQGVVRVKVSLSNQEAVITYQPYLI
QPEDLRDHVNDMGFEAAIKSKVAPLSLGPIDIERLQSTNPKRPLSSANQNENNSETLGHQGSHV
VTLQLRIDGMHCKSCVLNIEENIGQLLGVQSIQVSLENKTAQVKYDPSCTSPVALQRAIEALPP
GNFKVSLPDGAEGSGTDHRSSSSHSPGSPPRNQVQGTCSTTLIAIAGMTCASCVHSIEGMISQL
EGVQQISVSLAEGTATVLYNPSVISPEELRAAIEDMGFEASVVSESCSTNPLGNHSAGNSMVQT
TDGTPTSVQEVAPHTGRLPANHAPDILAKSPQSTRAVAPQKCFLQIKGMTCASCVSNIERNLQK
EAGVLSVLVALMAGKAEIKYDPEVIQPLEIAQFIQDLGFEAAVMEDYAGSDGNIELTITGMTCA
SCVHNIESKLTRINGITYASVALATSKALVKEDPEIIGPRDIIKIIEEIGFHASLAQRNPNAHH
LDHKMEIKQWKKSFLCSLVFGIPVMALMIYMLIPSNEPHQSMVLDHNIIPGLSILNLIFFILCT
FVQLLGGWYFYVQAYKSLRHRSANMDVLIVLATSIAYVYSLVILVVAVAEKAERSPVTFEDTPP
MLFVFIALGRWLEHLAKSKTSEALAKLMSLQATEATVVTLGEDNLIIREEQVPMELVQRGDIVK
VVPGGKFPVDGKVLEGNTMADESLITGEAMPVTKKPGSTVIAGSINAHGSVLIKATHVGNDTTL
AQIVKLVEEAQMSKAPIQQLADRESGYFVPFIIIMSTLTLVVWIVIGFIDFGVVQRYFPNPNKH
ISQTEVIIRFAFQTSITVLCIACPCSLGLATPTAVMVGTGVAAQNGILIKGGKPLEMAHKIKTV
MFDKTGTITHGVPRVMRVLLLGDVATLPLRKVLAVVGTAEASSEHPLGVAVTKYCKEELGTETL
GYCTDFQAVPGCGIGCKVSNVEGILAHSERPLSAPASHLNEAGSLPAEKDAVPQTFSVLIGNRE
WLRRNGLTISSDVSDAMTDHEMKGQTAILVAIDGVLCGMIAIADAVKQEAALAVHTLQSMGVDV
VLITGDNRKTARAIATQVGINKVFAEVLPSHKVAKVQELQNKGKKVAMVGDGVNDSPALAQADM
GVAIGTGTDVAIEAADVVLIRNDLLDVVASIHLSKRTVRRIRINLVLALIYNLVGIPIAAGVEM
PIGIVLQPWMGSAAMAASSVSVVLSSLQLKCYKKPDLERYEAQAHGHMKPLTASQVSVHIGMDD
RWRDSPRATPWDQVSYVSQVSLSSLTSDKPSRHSAAADDDGDKWSLLLNGRDEEQYI

6. Protein coding sequence of wildtype ATP7B mRNA, Homo sapiens ATPase copper transporting Beta (ATP7B), Transcript Variant 1, mRNA, NCBI Reference Sequence: NM_000053.4

atgcctg agcaggagag acagatcaca gccagagaag gggccagtcg gaaaatctta
tctaagcttt ctttgcctac ccgtgcctgg gaaccagcaa tgaagaagag
ttttgctttt gacaatgttg gctatgaagg tggtctggat ggcctgggcc
cttcttctca ggtggccacc agcacagtca ggatcttggg catgacttgc
cagtcatgtg tgaagtccat tgaggacagg atttccaatt tgaaaggcat
catcagcatg aaggtttccc tggaacaagg cagtgccact gtgaaatatg
tgccatcggt tgtgtgcctg caacaggttt gccatcaaat tggggacatg
ggcttcgagg ccagcattgc agaaggaaag gcagcctcct ggccctcaag
gtccttgcct gcccaggagg ctgtggtcaa gctccgggtg gagggcatga
cctgccagtc ctgtgtcagc tccattgaag gcaaggtccg gaaactgcaa
ggagtagtga gagtcaaagt ctcactcagc aaccaagagg ccgtcatcac
ttatcagcct tatctcattc agcccgaaga cctcagggac catgtaaatg
acatgggatt tgaagctgcc atcaagagca aagtggctcc cttaagcctg
ggaccaattg atattgagcg gttacaaagc actaacccaa agagaccttt
atcttctgct aaccagaatt ttaataattc tgagaccttg gggcaccaag
gaagccatgt ggtcaccctc caactgagaa tagatggaat gcattgtaag
tcttgcgtct tgaatattga agaaaatatt ggccagctcc taggggttca
aagtattcaa gtgtccttgg agaacaaaac tgcccaagta aagtatgacc
cttcttgtac cagcccagtg gctctgcaga gggctatcga ggcacttcca
cctgggaatt ttaaagtttc tcttcctgat ggagccgaag ggagtgggac
agatcacagg tcttccagtt ctcattcccc tggctcccca ccgagaaacc
aggtccaggg cacatgcagt accactctga ttgccattgc cggcatgacc
tgtgcatcct gtgtccattc cattgaaggc atgatctccc aactggaagg
ggtgcagcaa atatcggtgt ctttggccga agggactgca acagttcttt
ataatccctc tgtaattagc ccagaagaac tcagagctgc tatagaagac
atgggatttg aggcttcagt cgtttctgaa agctgttcta ctaaccctct
tggaaaccac agtgctggga attccatggt gcaaactaca gatggtacac
ctacatctgt gcaggaagtg gctccccaca ctgggaggct ccctgcaaac
catgccccgg acatcttggc aaagtcccca caatcaacca gagcagtggc
accgcagaag tgcttcttac agatcaaagg catgacctgt gcatcctgtg
tgtctaacat agaaaggaat ctgcagaaag aagctggtgt tctctccgtg
ttggttgcct tgatggcagg aaaggcagag atcaagtatg acccagaggt
catccagccc ctcgagatag ctcagttcat ccaggacctg ggttttgagg
cagcagtcat ggaggactac gcaggctccg atggcaacat tgagctgaca
atcacaggga tgacctgcgc gtcctgtgtc cacaacatag agtccaaact
cacgaggaca aatggcatca cttatgcctc cgttgccctt gccaccagca
aagcccttgt taagtttgac ccggaaatta tcggtccacg ggatattatc
aaaattattg aggaaattgg ctttcatgct tccctggccc agagaaaccc
caacgctcat cacttggacc acaagatgga aataaagcag tggaagaagt
ctttcctgtg cagcctggtg tttggcatcc ctgtcatggc cttaatgatc
tatatgctga tacccagcaa cgagccccac cagtccatgg tcctggacca
caacatcatt ccaggactgt ccattctaaa tctcatcttc tttatcttgt
gtacctttgt ccagctcctc ggtgggtggt acttctacgt tcaggcctac
aaatctctga gacacaggtc agccaacatg gacgtgctca tcgtcctggc
cacaagcatt gcttatgttt attctctggt catcctggtg gttgctgtgg
ctgagaaggc ggagaggagc cctgtgacat tcttcgacac gccccccatg
ctctttgtgt tcattgccct gggccggtgg ctggaacact tggcaaagag
caaaacctca gaagccctgg ctaaactcat gtctctccaa gccacagaag
ccaccgttgt gacccttggt gaggacaatt taatcatcag ggaggagcaa
gtccccatgg agctggtgca gcggggcgat atcgtcaagg tggtccctgg
gggaaagttt ccagtggatg ggaaagtcct ggaaggcaat accatggctg
atgagtccct catcacagga gaagccatgc cagtcactaa gaaacccgga
agcactgtaa ttgcggggtc tataaatgca catggctctg tgctcattaa
agctacccac gtgggcaatg acaccacttt ggctcagatt gtgaaactgg
tggaagaggc tcagatgtca aaggcaccca ttcagcagct ggctgaccgg
tttagtggat attttgtccc atttatcatc atcatgtcaa ctttgacgtt
ggtggtatgg attgtaatcg gttttatcga ttttggtgtt gttcagagat
actttcctaa ccccaacaag cacatctccc agacagaggt gatcatccgg
tttgctttcc agacgtccat cacggtgctg tgcattgcct gcccctgctc
cctggggctg gccacgccca cggctgtcat ggtgggcacc ggggtggccg
cgcagaacgg catcctcatc aagggaggca agcccctgga gatggcgcac
aagataaaga ctgtgatgtt tgacaagact ggcaccatta cccatggcgt
ccccagggtc atgcgggtgc tcctgctggg ggatgtggcc acactgcccc
tcaggaaggt tctggctgtg gtggggactg cggaggccag cagtgaacac
cccttgggcg tggcagtcac caaatactgt aaagaggaac ttggaacaga
gaccttggga tactgcacgg acttccaggc agtgccaggc tgtggaattg
ggtgcaaagt cagcaacgtg gaaggcatcc tggcccacag tgagcgccct
ttgagtgcac cggccagtca cctgaatgag gctggcagcc ttcccgcaga
aaaagatgca gtcccccaga ccttctctgt gctgattgga aaccgtgagt
ggctgaggcg caacggttta accatttcta gcgatgtcag tgacgctatg
acagaccacg agatgaaagg acagacagcc atcctggtgg ctattgacgg
tgtgctctgt gggatgatcg caatcgcaga cgctgtcaag caggaggctg
ccctggctgt gcacacgctg cagagcatgg gtgtggacgt ggttctgatc
acgggggaca accggaagac agccagagct attgccaccc aggttggcat
caacaaagtc tttgcagagg tgctgccttc gcacaaggtg gccaaggtcc
aggagctcca gaataaaggg aagaaagtcg ccatggtggg ggatggggtc
aatgactccc cggccttggc ccaggcagac atgggtgtgg ccattggcac
cggcacggat gtggccatcg aggcagccga cgtcgtcctt atcagaaatg
atttgctgga tgtggtggct agcattcacc tttccaagag gactgtccga
aggatacgca tcaacctggt cctggcactg atttataacc tggttgggat
acccattgca gcaggtgtct tcatgcccat cggcattgtg ctgcagccct
ggatgggctc agcggccatg gcagcctcct ctgtgtctgt ggtgctctca
tccctgcagc tcaagtgcta taagaagcct gacctggaga ggtatgaggc
acaggcgcat ggccacatga agcccctgac ggcatcccag gtcagtgtgc
acataggcat ggatgacagg tggcgggact cccccagggc cacaccatgg
gaccaggtca gctatgtcag ccaggtgtcg ctgtcctccc tgacgtccga
caagccatct cggcacagcg ctgcagcaga cgatgatggg gacaagtggt
ctctgctcct gaatggcagg gatgaggagc agtacatctg a

7. Polypeptide Sequence with the ATP7B,C9

MPEQERQITAREGASRKILSKLSLPTRAWEPAMKKSFAFDNVGYEGGLDGLGPSSQVATSTVRI
LGMTCQSCVKSIEDRISNLKGIISMKVSLEQGSATVKYVPSVVCLQQVCHQIGDMGFEASIAEG
KAASWPSRSLPAQEAVVKLRVEGMTCQSCVSSIEGKVRKLQGVVRVKVSLSNQEAVITYQPYLI
QPEDLRDHVNDMGFEAAIKSKVAPLSLGPIDIERLQSTNPKRPLSSANQNENNSETLGHQGSHV
VTLQLRIDGMHCKSCVLNIEENIGQLLGVQSIQVSLENKTAQVKYDPSCTSPVALQRAIEALPP
GNFKVSLPDGAEGSGTDHRSSSSHSPGSPPRNQVQGTCSTTLIAIAGMTCASCVHSIEGMISQL
EGVQQISVSLAEGTATVLYNPAVISPEELRAAIEDMGFEASVVSESCSTNPLGNHSAGNSMVQT
TDGTPTSLQEVAPHTGRLPANHAPDILAKSPQSTRAVAPQKCFLQIKGMTCASCVSNIERNLQK
EAGVLSVLVALMAGKAEIKYDPEVIQPLEIAQFIQDLGFEAAVMEDYAGSDGNIELTITGMTCA
SCVHNIESKLTRINGITYASVALATSKALVKFDPEIIGPRDIIKIIEEIGFHASLAQRNPNAHH
LDHKMEIKQWKKSFLCSLVFGIPVMALMIYMLIPSNEPHQSMVLDHNIIPGLSILNLIFFILCT
FVQLLGGWYFYVQAYKSLRHRSANMDVLIVLATSIAYVYSLVILVVAVAEKAERSPVTFEDTPP
MLFVFIALGRWLEHLAKSKTSEALAKLMSLQATEATVVTLGEDNLIIREEQVPMELVQRGDIVK
VVPGGKFPVDGKVLEGNTMADESLITGEAMPVTKKPGSTVIAGSINAHGSVLIKATHVGNDTTL
AQIVKLVEEAQMSKAPIQQLADRESGYFVPFIIIMSTLTLVVWIVIGFIDFGVVQRYFPNPNKH
ISQTEVIIRFAFQTSITVLCIACPCSLGLATPTAVMVGTGVAAQNGILIKGGKPLEMAHKIKTV
MFDKTGTITHGVPRVMRVLLLGDVATLPLRKVLAVVGTAEASSEHPLGVAVTKYCKEELGTETL
GYCTDFQAVPGCGIGCKVSNVEGILAHSERPLSAPASHLNEAGSLPAEKDAAPQTFSVLIGNRE
WLRRNGLTISSDVSDAMTDHEMKGQTAILVAIDGVLCGMIAIADAVKQEAALAVHTLQSMGVDV
VLITGDNRKTARAIATQVGINKVFAEVLPSHKVAKVQELQNKGKKVAMVGDGVNDSPALAQADM
GVAIGTGTDVAIEAADVVLIRNDLLDVVASIHLSKRTVRRIRINLVLALIYNLVGIPIAAGVEM
PIGIVLQPWMGSAAMAASSVSVVLSSLQLKCYKKPDLERYEAQAHGHMKPLTASQVSVHIGMDD
RWRDSPRATPWDQVSYVSQVSLSSLTSDKPSRHSAAADDDGDKWSLLLNGRDEEQYITETSQVA
PA

### 8. Example of Sequence of Different Polyadenylation Sites—SV40 Polyadenylation Sequence is Provided

ATGCTTTATTTGTGAAATTTGTGATGCTATTGCTTTATTTGTAACCATTA
TAAGCTGCAATAAACAAGTTAACAACAACAATTGCATTCATTTTATGTTT
CAGGTTCAGGGGGAGGTGTGGGAGGTTTTTTAAAGCAAGTAAAACCTCTA
CAAATGTGGTAAAATCCGATAAGGATCGATCCGGGC

9. Example of Sequences of the S/MAR Element. The Interferon-Beta Scaffold/Matrix Attachment Region is Provided Below.

AGTCAATATGTTCACCCCAAAAAAGCTGTTTGTTAACTTGCCAACCTCAT
TCTAAAATGTATATAGAAGCCCAAAAGACAATAACAAAAATATTCTTGTA
GAACAAAATGGGAAAGAATGTTCCACTAAATATCAAGATTTAGAGCAAAG
CATGAGATGTGTGGGGATAGACAGTGAGGCTGATAAAATAGAGTAGAGCT
CAGAAACAGACCCATTGATATATGTAAGTGACCTATGAAAAAAATATGGC
ATTTTACAATGGGAAAATGATGGTCTTTTTCTTTTTTAGAAAAACAGGGA
AATATATTTATATGTAAAAAATAAAAGGGAACCCATATGTCATACCATAC
ACACAAAAAAATTCCAGTGAATTATAAGTCTAAATGGAGAAGGCAAAACT
TTAAATCTTTTAGAAAATAATATAGAAGCATGCCATCAAGACTTCAGTGT
AGAGAAAAATTTCTTATGACTCAAAGTCCTAACCACAAAGAAAAGATTGT
TAATTAGATTGCATGAATATTAAGACTTATTTTTAAAATTAAAAAACCAT
TAAGAAAAGTCAGGCCATAGAATGACAGAAAATATTTGCAACACCCCAGT
AAAGAGAATTGTAATATGCAGATTATAAAAAGAAGTCTTACAAATCAGTA
AAAAATAAAACTAGACAAAAATTTGAACAGATGAAAGAGAAACTCTAAAT
AATCATTACACATGAGAAACTCAATCTCAGAAATCAGAGAACTATCATTG
CATATACACTAAATTAGAGAAATATTAAAAGGCTAAGTAACATCTGTGGC

### 10. Example of Stabilizing 3′UTR—Example of Albumin Sequence is Provided

CATCACATTTAAAAGCATCTCAGCCTACCATGAGAATAAGAGAAAGAAAA
TGAAGATCAAAAGCTTATTCATCTGTTTTTCTTTTTCGTTGGTGTAAAGC
CAACACCCTGTCTAAAAAACATAAATTTCTTTAATCATTTTGCCTCTTTT
CTCTGTGCTTCAATTAATAAAAAATGGAAAGAATCT

11. Example of Liver-Specific Promoter—hAPO-HCR Enhancer/hAAT Promoter

CCCTAAAATGGGCAAACATTGCAAGCAGCAAACAGCAAACACACAGCCCT
CCCTGCCTGCTGACCTTGGAGCTGGGGCAGAGGTCAGAGACCTCTCTGGG
CCCATGCCACCTCCAACATCCACTCGACCCCTTGGAATTTCGGTGGAGAG
GAGCAGAGGTTGTCCTGGCGTGGTTTAGGTAGTGTGAGAGGGGAATGACT
CCTTTCGGTAAGTGCAGTGGAAGCTGTACACTGCCCAGGCAAAGCGTCCG
GGCAGCGTAGGCGGGCGACTCAGATCCCAGCCAGTGGACTTAGCCCCTGT
TTGCTCCTCCGATAACTGGGGTGACCTTGGTTAATATTCACCAGCAGCCT
CCCCCGTTGCCCCTCTGGATCCACTGCTTAAATACGGACGAGGACAGG

### 12. Example of Cholangiocyte-Specific Promoter—CK19 Promoter

gcctgtaa tcccagcact ttggacggcc caggcgggtg gatcacttga ggtcaggagt
ttgagaccag cctggtcaac atggtgaaac cctgtctcta ctaaaaatac
aaaaattagc tgggcgtggt ggcgcgtgcc tgtaacccca gctactccgg
aggctgaggc aggagaatcg cttgaacccg ggagatggag gctgcagtgg
gccgagatca caccactgca ctccagtctg ggcgacagag actcgtctcc
aaaaaaacaa aaacaaaatc actgggtcag gggtgtgtag gaatatgacc
ccagagggac tgtaattccc agtggtgtca aactctgggt tgatcttaaa
gggtgaggct cagaatgtgg cttccaggga caggggtgtc caagtatgtc
cctgacgggg gaaaggccag aacagggtct gcagagagag agtgggggag
tgcgggtcgg agcttctgcg cggaccgggg cggggcacct ctggagggca
ggggcctctg gtctctggga ggggagggaa ttgaccaatg gggagagagc
ccatatttgc tctcaggagc ctgcaaattc ctcagggctc agatatccgc
ccctgacacc attcctccct tcccccctcc accggccgcg ggcataaaag
gcgccaggtg agggcctcgc cgctcctccc gcgaatcgca gcttctgaga
ccagggttgc tccgtccgtg ctccgcctcg cc

### 13. Example of Promoter Expressing in Cholangiocytes and Hepatocytes—CK18 Promoter

CAATAACAGTAAAAGGCAGTACGTAGCTTGTTGACTCCACATACTTTATTATAAAATACTGCCC
AACTTGACAGTTCTGGAATCCAGTGGGGGAATATAAAGGTGAAAGCAGGAGAGACCCCTCTGAC
TGGAACCTCTTACCTCCCAGAAGCCTTGTATGCAAAACCAGTGGGCATTCATTTGTATGTTATT
TTGCATCCCGTTTGCCTCCCAGCCTTCAGCAGGCCCCGACCCTCCCCTGGCCAGCTTCCACCCT
GACTGCCCCCTGGCTGGCTCCCATTGAGCACTGTGGGGCTCTCCCCACCATTAgGTGACAGATC
AgGAACAATCCAGGCTCAGGCTCTTTATCTGTGCTCTGCCTCCCACCTGGCAGGTCCACTGGCC
AGGCTTTTCCAGGGTCCCTTCTCTCYCAGGTCTGCCCTACTAtTTGTCCTCCCCTCCCCCTCAG
CTGGTAGCTCGATaAGAATCAATAGGTCCACTCCAGAGCAAAGAACACAGCCAAATGTGTCATA
CCAGGCCCTGCCAGAAAAACGAGCTGCTGGAGCTGACAAACTTGAAGGCCAAACACCTAAGGGT
TCCCCCCAACACTTCATTCAGCAGGGATGGTCATTCAGCTTCAGGGGGCAGGCAGCATGAAAGC
CTCCCTACCTCCATCCTTCTCACACAGAGGCTGGGGAGAGCATCTTGGAGGATGCAGTCCCCTG
GGGCCAGGCTTCTAATCCAGACAGCCCTTACAAGGGGGGACAGGGGAAGGACTGGCTTGGAGAA
AAGTCCTAGAAAAGAGGGGAGGGGCACTGGCCACCAGGGCTGGGTCGCTGCTATGATGGTCCTA
GGAGTGCCTGCCTGTCCTCTCAGGCCCCATGCGATGTAGGACACATTACTTTTATTTATTTATT
TATTTATTTATTTTGAGTCAGAGTTTCGCTCTGGTTGCCCAGGCTGGAGCGCGACGGCACGATC
TTGGCTCACTGCAACCTCTGCCTCCTGGGTTCAAGCGATTCTCCTGCCTCAGCCTCCTGAGTAG
CTGGGATTACAGGCACACACTGTGCCTGGTTAATTTTTGTATTTTTAGTAGAGAAGGGGTGTCA
CCATGTTGGTCAGGCTGGTCTCAAAtTTTTTTTTTTTTTTTTTTTTTTGAGACAGAGTCTTGCT
CTGTTGTCTAGGCTGGAGTGCAGTGGCATCGAACTCTTGACCTCAAGTGATCCACCCGCCTCGG
CCTCCCAAAGTGCTTGGATTACAGGCATGAGCCACTGTGCCCGGCGATGTGGGACACATTATCA
TCTCTGTGAGAGATTTTTGGTCTCTTTTGTCACCGCCCTTCTCTCCCAGCTCCTAGAACTGGGC
CTGGCTCACAGTAGGTGCTGAATGCATACTGGTTGAATTGTAAATGCTCAGGATTTGTTTAATT
AAGGATGCAGGAAAGGTGATATACCGGTGTGCAGAAGTCAGGATGCATTCCCTGTCCAAATCAC
AGTGTTCCACTGAGGCAAGGCCCTTGGGAGTGAGGTCGGGAGAGGGGAGGGTGGTGGAGGGGGC
TCAGAGACTGGGTTTGTTTTGGGGAGTCTGCACCTATTTGCTGAGTGAATGTATGTGTGTGTGC
ATTTGAGAGCACACCTCTGTATGATTCGGGTGTGAGTGTGTGTGAGGAAACGTGGGCAGGCGAG
GAGTGTTTGGGAGCCAGGTGCAGCTGGGGTGTGAGTGTGTAAGCAAGCAGCTATGAGGCTGGGC
ATTGCTTCTCCTCCTCTTCTCCAGCTCCCAGCCTTTCTTCCCCGGGACTCCTGGGGCTCCAGGA
TGCCCCCAAGATCCCCTCCACAAGTGGATAATTTGGGCTGCAGGTTAAGGACAGCTAGAGGGAC
TCACAGGCCATTCCACCCGCACACCACCAGACCCCCAAATTTCTTTTTTCTTTTTTTTTTTTTT
TTTTTTTTGAGACAGAGTCTCACTCTGTCGCCAGGCTGCAGTGGCGCGATCTCGGCTCACTGCA
ACCTCCGCCTCCCAGGTTCAAGCGATTCCCCTTCCTCAGCCTCCCAAGTAGCTGARACTACAGG
CGTGCACCATCACGTCCGGCTAATTTTTTGTATTTTAGTAGAGAGGGGGTTTCACCATGTTGGC
TAGGATGGTCTCGATCTCCTGACCTCGTGATCCGCCCACCTAGGCCTCCCAAAGTGCTGAGATT
ACAGGCGTGAGCCACTGCGCCCGGTCAAGACTCCCAAATTTCAAACTCGCCAGCACCTCCTCCA
CCTGGGGGAGAAGAGCATAATAACGTCATTTCCTGCCCTGAAAGCAGCCTCGAGGGCCAACAAC
ACCTGCTGTCCGTGTCCATGCCCGGTTGGCCACCCCGTTTCTGGGGGGTGAGCGGGGCTTGGCA
GGGCTGCGCGGAGGGCGCGGGGGTGGGGCCCGGGGCGGAGCGGCCCGGGGCGGAGGGCGCGGGC
TCCGAGCCGTCCACCTGTGGCTCCGGCTTCCGAAGCGGCTCCGGGGCGGGGGCGGGGCCTCACT
CTGCGATATAACTCGGGTCGCGCGGCTCGCGCAGGCCGCCACCGTCGTCCGCAAAGCCTGAGTC
CTGTCCTTTCTCTCACGCGTCAGGTAAAGGGGTAGGAGGGACCTCAACTCCCCAGCCTTGTCTG
ACCCCTCCCAATTATTACACTCCTTTGCCTCTTCCGTCATTCCCATAACCACCCAACCCTACTC
CACCGGGAGGGGGTTGGGCATACCTGGATTTCCATCCGCGCACCTAGCCACAGGGTCCCTAAGA
GCAGCAGCTAGGCATGGGAGGGCTCTTTCCCAGGAGAGAGGGGGAAGGGGACAGGGTTGAGAGC
TTTACAGAGGAAGTGGACAGCATGGAGGGAGGTAAGGAAAGGCCTGTAAAGAGGAGGAGACACT
GGCTCTGGCGGAATGGGGACTATTGGAGGGTTAAGCGGATGTGGCTAAGGCTGAGTCATCTAGG
AGTAAACAAGAGGCCTTCCTTTGGGAGGAGCCAATCCAGGGTGTAGGGGGCCCAGAGTGACCAG
GTGCACTAGGGAAAAAATGCCAGGAGAGGGCCAGGAAGAGGACTTGTTAGTAGCGACTCACTTC
TGGGCAGGCAGGCCAGCCAGCTAGCCAGCCTGCTGAGGCTTCCCAAGAGGGGCAGAGTGCTGGG
ATCTGGGAATCCAGGAAAGGAGGGAATGGGGTGGGGCTAGATGAAAAGGGATAGGTGTCCAGGG
AGAGCCTCTGGCTATTCCTGGGACCAGGAAGTTTTCACTAGGATACATAACACTTTTTACACAC
TCACCCCACCCATCCCTGGCTTTCTATTCATGGAACAACCTCTCTCT

### 14. PT-LP1-ATP7B: Transposon Expressing ATP7B Under Hepatocyte-Specific Expression

TTAACCCTAGAAAGATAGTCTGCGTAAAATTGACGCATGCATTCTTGAAATATTGCTCTCTCTT
TCTAAATAGCGCGAATCCGTCGCTGTGCATTTAGGACATCTCAGTCGCCGCTTGGAGCTCCCGT
GAGGCGTGCTTGTCAATGCGGTAAGTGTCACTGATTTTGAACTATAACGACCGCGTGAGTCAAA
ATGACGCATGATTATCTTTTACGTGACTTTTAAGATTTAACTCATACGATAATTATATTGTTAT
TTCATGTTCTACTTACGTGATAACTTATTATATATATATTTTCTTGTGGAGGGGCTAGCTCGTG
ACCCCTAAAATGGGCAAACATTGCAAGCAGCAAACAGCAAACACACAGCCCTCCCTGCCTGCTG
ACCTTGGAGCTGGGGCAGAGGTCAGAGACCTCTCTGGGCCCATGCCACCTCCAACATCCACTCG
ACCCCTTGGAATTTCGGTGGAGAGGAGCAGAGGTTGTCCTGGCGTGGTTTAGGTAGTGTGAGAG
GGGAATGACTCCTTTCGGTAAGTGCAGTGGAAGCTGTACACTGCCCAGGCAAAGCGTCCGGGCA
GCGTAGGCGGGCGACTCAGATCCCAGCCAGTGGACTTAGCCCCTGTTTGCTCCTCCGATAACTG
GGGTGACCTTGGTTAATATTCACCAGCAGCCTCCCCCGTTGCCCCTCTGGATCCACTGCTTAAA
TACGGACGAGGACAGGGCCCTGTCTCCTCAGCTTCAGGCACCACCACTGACCTGGGACAGTGAA
TCCGGACTCTAAGGTAAATATAAAATTTTTAAGTGTATAATGTGTTAAACTACTGATTCTAATT
GTTTCTCTCTTTTAGATTCCAACCTTTGGAACTGAATTctagACCACCATGCCTGAACAGGAGA
GACAGATCACAGCCAGAGAAGGGGCCAGTCGGAAAATCTTATCTAAGCTTTCTTTGCCTACCCG
TGCCTGGGAACCAGCAATGAAGAAGAGTTTTGCTTTTGACAATGTTGGCTATGAAGGTGGTCTG
GATGGCCTGGGCCCTTCTTCTCAGGTGGCCACCAGCACAGTCAGGATCTTGGGCATGACTTGCC
AGTCATGTGTGAAGTCCATTGAGGACAGGATTTCCAATTTGAAAGGCATCATCAGCATGAAGGT
TTCCCTGGAACAAGGCAGTGCCACTGTGAAATATGTGCCATCGGTTGTGTGCCTGCAACAGGTT
TGCCATCAAATTGGGGACATGGGCTTCGAGGCCAGCATTGCAGAAGGAAAGGCAGCCTCCTGGC
CCTCAAGGTCCTTGCCTGCCCAGGAGGCTGTGGTCAAGCTCCGGGTGGAGGGCATGACCTGCCA
GTCCTGTGTCAGCTCCATTGAAGGCAAGGTCCGGAAACTGCAAGGAGTAGTGAGAGTCAAAGTC
TCACTCAGCAACCAAGAGGCCGTCATCACTTATCAGCCTTATCTCATTCAGCCCGAAGACCTCA
GGGACCATGTAAATGACATGGGATTTGAAGCTGCCATCAAGAGCAAAGTGGCTCCCTTAAGCCT
GGGACCAATTGATATTGAGCGGTTACAAAGCACTAACCCAAAGAGACCTTTATCTTCTGCTAAC
CAGAATTTTAATAATTCTGAGACCTTGGGGCACCAAGGAAGCCATGTGGTCACCCTCCAACTGA
GAATAGATGGAATGCATTGTAAGTCTTGCGTCTTGAATATTGAAGAAAATATTGGCCAGCTCCT
AGGGGTTCAAAGTATTCAAGTCTCCTTGGAGAACAAAACTGCCCAAGTAAAGTATGACCCTTCT
TGTACCAGCCCAGTGGCTCTGCAGAGGGCTATCGAGGCACTTCCACCTGGGAATTTTAAAGTTT
CTCTTCCTGATGGAGCCGAAGGGAGTGGGACAGATCACAGGTCTTCCAGTTCTCATTCCCCTGG
CTCCCCACCGAGAAACCAGGTCCAGGGCACATGCAGTACCACTCTGATTGCCATTGCCGGCATG
ACCTGTGCATCCTGTGTCCATTCCATTGAAGGCATGATCTCCCAACTGGAAGGGGTGCAGCAAA
TATCGGTGTCTTTGGCCGAAGGGACTGCAACAGTTCTTTATAATCCCGCTGTAATTAGCCCAGA
AGAACTCAGAGCTGCTATAGAAGACATGGGATTTGAGGCTTCAGTCGTTTCTGAAAGCTGTTCT
ACTAACCCTCTTGGAAACCACAGTGCTGGGAATTCCATGGTGCAAACTACAGATGGTACACCTA
CATCTCTGCAGGAAGTGGCTCCCCACACTGGGAGGCTCCCTGCAAACCATGCCCCGGACATCTT
GGCAAAGTCCCCACAATCAACCAGAGCAGTGGCACCGCAGAAGTGCTTCTTACAGATCAAAGGC
ATGACCTGTGCATCCTGTGTGTCTAACATAGAAAGGAATCTGCAGAAAGAAGCTGGTGTTCTCT
CCGTGTTGGTTGCCTTGATGGCAGGAAAGGCAGAGATCAAGTATGACCCAGAGGTCATCCAGCC
CCTCGAGATAGCTCAGTTCATCCAGGACCTGGGTTTTGAGGCAGCAGTCATGGAGGACTACGCA
GGCTCCGATGGCAACATTGAGCTGACAATCACAGGGATGACCTGCGCGTCCTGTGTCCACAACA
TAGAGTCCAAACTCACGAGGACAAATGGCATCACTTATGCCTCCGTTGCCCTTGCCACCAGCAA
AGCCCTTGTTAAGTTTGACCCGGAAATTATCGGTCCACGGGATATTATCAAAATTATTGAGGAA
ATTGGCTTTCATGCTTCCCTGGCCCAGAGAAACCCCAACGCTCATCACTTGGACCACAAGATGG
AAATAAAGCAGTGGAAGAAGTCTTTCCTGTGCAGCCTGGTGTTTGGCATCCCTGTCATGGCCTT
AATGATCTATATGCTGATACCCAGCAACGAGCCCCACCAGTCCATGGTCCTGGACCACAACATC
ATTCCAGGACTGTCCATTCTAAATCTCATCTTCTTTATCTTGTGTACCTTTGTCCAGCTCCTCG
GTGGGTGGTACTTCTACGTTCAGGCCTACAAATCTCTGAGACACAGGTCAGCCAACATGGACGT
GCTCATCGTCCTGGCCACAAGCATTGCTTATGTTTATTCTCTGGTCATCCTGGTGGTTGCTGTG
GCTGAGAAGGCGGAGAGGAGCCCTGTGACATTCTTCGACACGCCCCCCATGCTCTTTGTGTTCA
TTGCCCTGGGCCGGTGGCTGGAACACTTGGCAAAGAGCAAAACCTCAGAAGCCCTGGCTAAACT
CATGTCTCTCCAAGCCACAGAAGCCACCGTTGTGACCCTTGGTGAGGACAATTTAATCATCAGG
GAGGAGCAAGTCCCCATGGAGCTGGTGCAGCGGGGCGATATCGTCAAGGTGGTCCCTGGGGGAA
AGTTTCCAGTGGATGGGAAAGTCCTGGAAGGCAATACCATGGCTGATGAGTCCCTCATCACAGG
AGAAGCCATGCCAGTCACTAAGAAACCCGGAAGCACTGTAATTGCGGGGTCTATAAATGCACAT
GGCTCTGTGCTCATTAAAGCTACCCACGTGGGCAATGACACCACTTTGGCTCAGATTGTGAAAC
TGGTGGAAGAGGCTCAGATGTCAAAGGCACCCATTCAGCAGCTGGCTGACCGGTTTAGTGGATA
TTTTGTCCCATTTATCATCATCATGTCAACTTTGACGTTGGTGGTATGGATTGTAATCGGTTTT
ATCGATTTTGGTGTTGTTCAGAAATACTTTCCTAACCCCAACAAGCACATCTCCCAGACAGAGG
TGATCATCCGGTTTGCTTTCCAGACGTCCATCACGGTGCTGTGCATTGCCTGCCCCTGCTCCCT
GGGGCTGGCCACGCCCACGGCTGTCATGGTGGGCACCGGGGTGGCCGCGCAGAACGGCATCCTC
ATCAAGGGAGGCAAGCCCCTGGAGATGGCGCACAAGATAAAGACTGTGATGTTTGACAAGACTG
GCACCATTACCCATGGCGTCCCCAGGGTCATGCGGGTGCTCCTGCTGGGGGATGTGGCCACACT
GCCCCTCAGGAAGGTTCTGGCTGTGGTGGGGACTGCGGAGGCCAGCAGTGAACACCCCTTGGGC
GTGGCAGTCACCAAATACTGTAAAGAGGAACTTGGAACAGAGACCTTGGGATACTGCACGGACT
TCCAGGCAGTGCCAGGCTGTGGAATTGGGTGCAAAGTCAGCAACGTGGAAGGCATCCTGGCCCA
CAGTGAGCGCCCTTTGAGTGCACCGGCCAGTCACCTGAATGAGGCTGGCAGCCTTCCCGCAGAA
AAAGATGCAGCCCCCCAGACCTTCTCTGTGCTGATTGGAAACCGTGAGTGGCTGAGGCGCAACG
GTTTAACCATTTCTAGCGATGTCAGTGACGCTATGACAGACCACGAGATGAAAGGACAGACAGC
CATCCTGGTGGCTATTGACGGTGTGCTCTGTGGGATGATCGCAATCGCAGACGCTGTCAAGCAG
GAGGCTGCCCTGGCTGTGCACACGCTGCAGAGCATGGGTGTGGACGTGGTTCTGATCACGGGGG
ACAACCGGAAGACAGCCAGAGCTATTGCCACCCAGGTTGGCATCAACAAAGTCTTTGCAGAGGT
GCTGCCTTCGCACAAGGTGGCCAAGGTCCAGGAGCTCCAGAATAAAGGGAAGAAAGTCGCCATG
GTGGGGGATGGGGTCAATGACTCCCCGGCCTTGGCCCAGGCAGACATGGGTGTGGCCATTGGCA
CCGGCACGGATGTGGCCATCGAGGCAGCCGACGTCGTCCTTATCAGAAATGATTTGCTGGATGT
GGTGGCTAGCATTCACCTTTCCAAGAGGACTGTCCGAAGGATACGCATCAACCTGGTCCTGGCA
CTGATTTATAACCTGGTTGGGATACCCATTGCAGCAGGTGTCTTCATGCCCATCGGCATTGTGC
TGCAGCCCTGGATGGGCTCAGCGGCCATGGCAGCCTCCTCTGTGTCTGTGGTGCTCTCATCCCT
GCAGCTCAAGTGCTATAAGAAGCCTGACCTGGAGAGGTATGAGGCACAGGCGCATGGCCACATG
AAGCCCCTGACGGCATCCCAGGTCAGTGTGCACATAGGCATGGATGACAGGTGGCGGGACTCCC
CCAGGGCCACACCATGGGACCAGGTCAGCTATGTCAGCCAGGTGTCGCTGTCCTCCCTGACGTC
CGACAAGCCATCTCGGCACAGCGCTGCAGCAGACGATGATGGGGACAAGTGGTCTCTGCTCCTG
AATGGCAGGGATGAGGAGCAGTACATCTGATGAAgatcTCATCACATTTAAAAGCATCTCAGCC
TACCATGAGAATAAGAGAAAGAAAATGAAGATCAAAAGCTTATTCATCTGTTTTTCTTTTTCGT
TGGTGTAAAGCCAACACCCTGTCTAAAAAACATAAATTTCTTTAATCATTTTGCCTCTTTTCTC
TGTGCTTCAATTAATAAAAAATGGAAAGAATCTATGCTTTATTTGTGAAATTTGTGATGCTATT
GCTTTATTTGTAACCATTATAAGCTGCAATAAACAAGTTAACAACAACAATTGCATTCATTTTA
TGTTTCAGGTTCAGGGGGAGGTGTGGGAGGTTTTTTAAAGCAAGTAAAACCTCTACAAATGTGG
TAAAATCCGATAAGGATCGATCCGGGCTCGAGTTTTGTTACTTTATAGAAGAAATTTTGAGTTT
TTGTTTTTTTTTAATAAATAAATAAACATAAATAAATTGTTTGTTGAATTTATTATTAGTATGT
AAGTGTAAATATAATAAAACTTAATATCTATTCAAATTAATAAATAAACCTCGATATACAGACC
GATAAAACACATGCGTCAATTTTACGCATGATTATCTTTAACGTACGTCACAATATGATTATCT
TTCTAGGGTTAA

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

The above-mentioned and other features and advantages of the present disclosure will be better understood when reading the following detailed description taken together with the following drawings in which:

FIGS. 1A-1C depict construction and functional validation of plasmid expressing hATP7B with C9-tag. FIG. 1A shows a C9-tag, corresponding to the carboxy-terminal 9 amino acids of bovine rhodopsin was added to the carboxy-terminus of human ATP7B located in the intracellular surface. FIG. 1B shows the gene, ATP7B,C9, was cloned into a plasmid DNA vector, pT-LP1, with a liver-specific LP1 promoter driving expression between the terminal repeats of piggyBac transposon, which facilitate integration. SV40 polyadenylation sequence is not shown. Total pDNA size is 8.6 kB, including the bacterial backbone. FIG. 1C shows the intracellular movement of hATP7B,C9 in response to different copper levels (basal, low, and high) inside cells was examined. In cells lacking native ATP7B expression, antibody staining for ATP7B and C9 tag (1D4 antibody) efficiently co-localized, with ATP7B,C9 moving out of the trans-golgi network (TGN) in response to high copper levels. FIG. 1D shows ATP7B,C9 copper transporting activity was examined in cells in cells lacking native ATP7B expression. Co-transfection of ATP7B,C9 or an inactive ATP7B (D1027A) and a plasmid encoding copper-dependent tyrosinase is performed, with successful eumelanin formation (black pigment) only in cells where copper can be transported into the TGN and vesicles.

FIGS. 2A-2E show the results of hydrodynamic tail vein injection of pT-LP1-hATP7B,C9 results in reduction of liver injury and hepatic copper content in WD mice. FIG. 2A shows the experimental plan, wherein female WD mice were hydrodynamically injected with 1 μg of pT-LP1-hATP7B,C9 and 1 μg pCMV-hyPBase, the latter for integration. Heterozygous mice were also injected as a control, and all mice were bleed and harvested at 20 weeks for further analysis. Serum obtained at 20 weeks for un-injected heterozygous mice, un-injected ATP7B KO mice, and treated ATP7B KO mice were compared for ALT (FIG. 2B), AST (FIG. 2C), and LDH (FIG. 2D). FIG. 2E shows that the hepatic copper content was measured in the same mice, with significant reduction in the treatment group. Statistical significance was calculated with unpaired, parametric t-tests, *p<0.05, **p<0.005, ***p<0.0005.

FIGS. 3A-3D depict hydrodynamic tail vein injection achieves transfection of hepatocytes with ATP7B,C9 without significant expansion over time. FIG. 3A shows immunohistochemical staining for ATP7B,C9 for treated WD mice reveals positive hepatocytes with variable morphology and staining intensity. FIG. 3B shows quantification of 1 ug of ATP7B,C9 plasmid transfection in ATP7B KO mice and Heterozygous mice by percent area stained, measured at 20 weeks of age (8 random 20× fields measured for each mouse, n=3 mice each group). FIG. 3C shows that 10 μg of reporter plasmid, pCMV-GFP, was injected into WD and Het mice by HTVI, and the % stained area compared at 3 days post-injection. FIG. 3D shows H&E staining for female heterozygous, KO untreated, and KO treated mice are depicted, showing minimal differences. Statistical significance was calculated with unpaired, parametric t-tests, *p<0.05, **p<0.005, ***p<0.0005.

FIGS. 4A-4E show that biliary hydrodynamic injection can successfully mediate hATP7B,C9 expression in pig liver. FIG. 4A shows that ERCP can be used to access the common hepatic duct, with fluoroscopy verifying catheter position and branching into the liver prior to injection. FIG. 4B demonstrates that harvested pig liver shows no abnormalities from injection and no rupture of bile ducts. Sampling of the right lateral, right medial, left medial, and later lateral liver lobes was conducted. FIG. 4C shows the immunohistochemistry for C9-tag in pig liver reveals abundant hATP7B,C9 positive hepatocytes, which were located across all three zones. Rare liver lobules had transfection that exceeded over 80% of hepatocytes. FIG. 4D shows immunofluorescent staining for ATP7B,C9 in pigs demonstrates localization of ATP7B,C9 inside pig hepatocytes. This pattern was similar to ATP7B staining for native pig ATP7B. FIG. 4E shows quantification of immunostaining in pigs dosed at 10 mg pDNA. Percent area was calculated among liver lobules, with 8 liver lobules averaged.

FIGS. 5A-E shows serum chemistries reported for WD mice treated with 1 μg of pT-LP1-hATP7B,C9 by hydrodynamic injection. Mice were bled at 20 weeks age, and six weeks post-DNA injection. ATP7B KO mice were treated with 1 μg of pT-LP1-hATP7B,C9, while control groups of untreated heterozygous and KO mice were also analyzed. Albumin (FIG. 5A), Alkaline phosphatase (FIG. 5B), total bilirubin (FIG. 5C), total protein (FIG. 5D), and glucose (FIG. 5E) are presented. Statistical significance was calculated with unpaired, parametric t-tests, *p<0.05, **p<0.005, ***p<0.0005, n.s. non-significant.

FIGS. 6A-D shows hydrodynamic injection of 25 μg of pT-LP1-hATP7B,C9 into WD mice did not yield improvements in liver injury. In FIG. 6A the experimental plan is depicted: male and female WD mice were hydrodynamically injected with 25 μg of pT-LP1-hATP7B,C9 and 10 μg pCMV-hyPBase, the latter for integration. Mice were injected prior to WD liver phenotype development, and mice were harvested at 20 weeks for further analysis. The same female heterozygous and KO mice depicted in FIG. 2 are presented here as control groups for comparison. Serum chemistries for ALT (FIG. 6B), AST (FIG. 6C), and LDH (FIG. 6D) are presented. Statistical significance was calculated with unpaired, parametric t-tests, *p<0.05, **p<0.005, ***p<0.0005, n.s. non-significant.

FIGS. 7A-C shows hydrodynamic injection of 25 μg of pT-LP1-hATP7B,C9 mediates high-level expression in mouse liver, but expression is lost over time. FIG. 7A shows that hydrodynamic injection of 25 μg of pT-LP1-hATP7B,C9 and 10 μg pCMV-hyPBase mediates high-level expression and transfection efficiency in mice 4 days after transfection. FIG. 7B shows quantification of the percent area stained for the mice dosed with 25 μg of pT-LP1-hATP7B,C9, four days post-injection. 8 random 20× fields, n=2 mice presented. FIG. 7C shows mice that are 12 weeks post-hydrodynamic injection of 25 μg of pT-LP1-hATP7B,C9 and 10 μg pCMV-hyPBase display were examined by immunofluorescence for ATP7B,C9 expression using the 1D4 antibody and anti-mouse FITC secondary. Mice harvested 4 days post-injection served as a control.

FIGS. 8A-D shows hydrodynamic injection of 5 μg of pT-LP1-hATP7B,C9 into WD mice did not yield improvements in liver injury. In FIG. 8A the experimental plan is depicted: male and female WD mice were hydrodynamically injected with 5 μg of pT-LP1-hATP7B,C9 and 5 μg pCMV-hyPBase, the latter for integration. Mice were injected prior to WD liver phenotype development, and mice were harvested at 20 weeks for further analysis. The same female heterozygous and KO mice depicted in FIG. 2 are presented here as control groups for comparison. Serum chemistries for ALT (FIG. 8B), AST (FIG. 8C), and LDH (FIG. 8D) are presented. Statistical significance was calculated with unpaired, parametric t-tests, *p<0.05, **p<0.005, ***p<0.0005, n.s. non-significant.

### DETAILED DESCRIPTION

The present disclosure relates to compositions and methods for treating Wilson's disease. More particularly, the present disclosure relates to compositions and methods for treating Wilson's disease by gene therapy. As described in detail below, the present disclosure is based, at least in part, on the surprising discovery that non-viral modification of hepatocytes with ATP7B gene in Wilson's Disease (WD) liver can lead to expansion of those hepatocytes through a proliferative advantage. This phenomenon had not previously observed or reported in other gene therapy or cell therapy strategies. This feature was accomplished through stable expression of ATP7B in host hepatocytes, which can be accomplished through transposons or other integrating strategies. Selection was also facilitated through even dispersion of gene vector throughout liver tissue with hydrodynamic injection. The disclosure further describes vectors that also yield expression in cholangiocytes in addition to hepatocytes, which are also important in Wilson's Disease pathology but not targeted in other gene therapy strategies. Targeting cholangiocytes further increases export of copper into the bile. This is accomplished through the use of novel synthetic promoters that drive expression in both cell types. These synthetic promoters consist of tandem arrangements of hepatocyte and cholangiocyte-specific promoters not naturally occurring, which are empirically observed to not interfere with each other and achieve expression in both cell types. In other cases, promoters that express naturally in both cell types are selected for ATP7B expression.

### Overview

Wilson disease (WD) is a monogenic liver disease that results in the buildup of toxic levels of copper in different tissues, primarily affecting the liver and brain (Członkowska et al 2018). WD is caused by various mutations in ATP7B, which codes for a copper transporting transmembrane protein. The WD-causing mutations in ATP7B disrupt protein stability, intracellular localization, and copper transporting function. WD is a autosomal recessive disorder, and the commonly observed compound heterozygous mutations produce a broad spectrum of disease-onset and manifestations (Członkowska et al 2018). The liver disease can eventually progress to cirrhosis and liver failure, while the brain toxicity can result in neuropsychiatric symptoms. Treatments for WD include penicillamine and trientine, which are copper chelating agents that facilitates copper removal from the body, reducing tissue damage. However, penicillamine can have significant toxicities resulting in poor compliance among WD patients (Masełbas et al 2019). Lack of compliance can lead to ongoing copper toxicities with patients continuing to progress in disease pathology.

Given that WD is a single-gene disorder, it is an attractive target for gene therapy. While ATP7B is expressed in many different tissues, the replacement of ATP7B gene has primarily focused on the liver. Liver plays a central role in the whole-body copper homeostasis and ATP7B is essential for this liver function: ATP7B facilitates the delivery of copper to ceruloplasmin (the major copper containing protein in a serum) and exports excess copper into bile. Liver transplants are able to cure WD in patients developing liver failure, and recent clinical studies suggest that they may even improve neuropsychiatric symptoms (Poujois et al 2020). Liver transplants are, however, a risky procedure with numerous comorbidities and require the lifelong maintenance of immunosuppression to ensure graft survival. The limited availability of organs and these risk and morbidities of transplant limit the use of liver transplantation for WD treatment.

### ATP7B Molecular Genetics

Wilson's Disease (WD) manifestations range from mild hepatic inflammation and tremor to cirrhosis, fulminant liver failure, depression, and psychotic episodes. The exact cause of this phenotypic variability remains poorly understood. Over 600 WD-causing mutations have been identified in ATP7B and increasing number of those mutations has been characterized. These studies revealed a spectrum of biochemical and cellular effects, ranging from a complete loss of ATP7B expression and function to potentially milder effects on ATP7B trafficking or stability. However, strong correlations between mutations and associated phenotypes have not been observed, suggesting existence of additional, modifying factors. (Metallomics. 2019 Jun. 19; 11(6): 1128-1139).

The WD gene ATP7B (adenosine triphosphatase, copper transporting, beta polypeptide) is expressed in the liver and brain. There are more alternatively spliced variants of ATP7B in the brain than in the liver. The most abundant form in the liver contains all the exons, whereas splice variants in the brain have several combinations of skipped exons. Tissue-specific mechanisms regulate alternative splicing of ATP7B in the liver and brain. For example, alternative splicing of exon 12 occurs in the brain but not in the liver. It is not known whether these splice variants retain their biological function. A mutation in Wilson's Disease patient that yields an alternatively spliced and removed exon 12 suggests that domain is essential for liver function (Hepatology. 2010 November; 52(5):1662-70).

More recently, a total of 995 mutations in ATP7B were annotated in the Human Gene Mutation Database (HGMD)1, in which 67 splice variants located in the non-coding region were included. No synonymous variants were reported in the HGMD. Synonymous variants are often considered to be neutral because they do not change the amino acid coding. However, recent studies have shown that synonymous/missense mutations can lead to abnormal splicing of mRNA by disrupting splicing regulatory elements (SREs), which act as a new, gradually recognized pathogenic mechanism in genetic diseases. This mechanism has been reported in Gitelman syndrome, progressive familial intrahepatic cholestasis, Netherton syndrome, and other genetic disorders (Byrne et al., 2009; Dal Mas et al., 2015; Takeuchi et al., 2015). A previous study demonstrated that the synonymous variant (c.4014T>A) and missense variant (c.2755C>G/p.R919G) in WD patients resulted in exon skipping in pre-mRNA splicing, leading to protein truncation and WD (Wang et al., 2018). (Front Pediatr. 2018; 6: 106)

Gene therapy could solve the issues of liver transplant by directly delivering the gene into the patient's hepatocytes without the need for long-term immunosuppression. Gene therapy has been explored and showed promise in WD mouse models. Using adeno-associated viruses (AAV) as the delivery vehicle for ATP7B, significant improvements in liver enzyme function and reductions in copper in the liver and other tissues were achieved (Murillo et al 2016; Murillo et al 2019). However, a chief problem using AAV has been the size limitation of 4.8 kB of the vector; the ATP7B cDNA itself is ~4.4 kB leaving not enough room for the gene expression elements and AAV ITR's. Prior art studies have utilized truncated forms of ATP7B with several of the metal binding domains deleted (Leng et al 2019), but these truncated forms may have altered protein function and regulation compared to the full-length protein. In order to deliver full length ATP7B with regulatory elements, non-viral approaches without size restriction could be utilized. Hydrodynamic gene delivery can deliver naked plasmid DNA (pDNA) directly into hepatocytes in mice (Liu et al 1999) but has not previously been explored to deliver ATP7B for WD gene therapy.

The present disclosure provides methods and compositions for an effective gene therapy for WD. First, the disclosure provides a technique of non-viral hydrodynamic delivery to deliver the full-length ATP7B cDNA into hepatocytes. Second, the disclosure provides a vector composition and method to endow hepatocytes expressing ATP7B with a proliferative advantage over non-transfected hepatocytes, which is optionally aided by using a transposon system to mediate integration into the hepatocyte genome. Third, the disclosure provides a method to achieve efficient ATP7B expression in the liver of human-sized large animals at clinically relevant, disease-modifying levels. No previous gene delivery studies have provided methods and compositions that achieved hATP7B gene delivery into large animals. The present disclosure provides a method of efficient ATP7B expression from a non-viral vector that paves the way for clinical translation.

Gene therapy represents a potential cure for Wilson's disease, which is caused by mutations of the ATP7B gene leading to decreased function of the protein in transporting copper. The lack of normal ATP7B function leads to the buildup of copper in the liver, brain and other tissues, eventually leading to organ damage in these tissues and various clinical signs and symptoms.

Previous attempts at gene therapy for Wilson's Disease have focused the delivery of ATP7B with adeno-associated virus (AAV) vectors. AAV vectors have size limitations of around 4.7-8 kb in total. This size limitation makes it challenging to use the platform as an ATP7B delivery vehicle, given that ATP7B cDNA has a vector size of 4.4 kb for the protein coding sequence.

With the AAV ITR's being 0.2-0.3 kb total in size, this leaves no room for promoter and polyadenylation sequences if the full-length ATP7B is used. While a previous investigation sought to deliver full-length ATP7B, the yield of AAV was very low, although enough vector was recovered to perform treatment experiments in mice (J Hepatol. 2016 February; 64(2):419-426.).

In a follow up study, the investigators shifted to the use of deletion variants of ATP7B in order to create extra room for promoter and polyadenylation sequences and to increase AAV titer yield by ensuring total vector length below 4.8 kb. It remains to be conclusively proven that these deletion variants have the same function long-term as full-length ATP7B. It should be noted that microdystrophin genes for Duchenne's muscular dystrophy proved effective in mouse models after AAV gene therapy but have proven to be ineffective in actual human patients (https://www.evaluate.com/vantage/articles/news/trial-results/gene-therapy-trial-fails-rectify-sareptas-sorry-record). Importantly, even with these changes to use a mini ATP7B to ensure length under 4.8 kb and high viral vector yield, the promoters utilized consist of small, liver-specific promoters driven by ubiquitous high-level expression and lack regulation of ATP7B in response to copper metabolism.

In summary, the prior art presents several limitations. Full-length ATP7B protein is not being utilized, promoter elements to do not feature native regulation of ATP7B expression in sensing copper status. Another important limitation is that AAV-based platforms represent the disadvantages of immune responses against the capsid, preventing redosing of AAV and causing acute toxicity. Many patients are excluded from trials because of pre-existing antibodies against the AAV capsid. Post-administration, T cell responses develop against the capsid and can destroy transgene reduced cells. Eventually, antibody responses develop against the AAV delivered, which prevents any redosing. The prior art also fails to address the loss of DNA vector over time with cell division of hepatocytes, which is inevitable given the chronic inflammation in Wilson's disease patients.

The current technology provides numerous improvements on the prior art. Given the liver damage and hepatocyte turnover in Wilson's Disease patients, there has been speculated in the literature a possibility of a proliferative advantage of hepatocytes expressing ATP7B given that they will be protected from copper overload.

A major feature of the technology is affording hepatocytes expressing ATP7B to have a proliferative advantage without requiring the use of liver toxins or partial hepatectomy. The latter strategies had been necessary in the prior art for cell therapy strategies (Mol Ther. 2001 March; 3(3):302-9)., suggesting a very weak or minimal proliferative advantage for ATP7B expressing cells. Moreover, such proliferative advantage has not been observed or described in prior AAV or lentiviral studies (Roybal et al 2012). Together, the disclosure outlines strategies for a non-viral approach to endow hepatocytes with ATP7B expression distributed initially throughout the liver, and at expression levels such that these cells will increase in number over time, as compared to a liver that did not have Wilson's Disease.

The disclosure describes optimal design of a DNA vector for use in hydrodynamic gene delivery into the liver of Wilson's disease patients. Hydrodynamic gene delivery has been described for several other rare monogenic diseases in the prior art in mouse models, but has not been previously elucidated for Wilson's disease, either in mouse models, large mammals, or human patients. Thus, the optimal DNA vector design remains uncertain for Wilson's Disease gene therapy. In particular, the potential for toxicity and suitability of expression from a non-viral vector has not been elucidated. The current disclosure describes a DNA vector for expressing ATP7B with several important improvements, which will be summarized as follows:

The current disclosure envisions the delivery of a naked DNA molecule into hepatocytes of an individuals with Wilson's disease, with the DNA molecule entering into cell and eventually the nucleus of hepatocytes yielding expression. The DNA molecule in most embodiments will be a circular DNA molecule, itself either a plasmid DNA molecule, or derived from a plasmid DNA molecule. In other embodiments, the DNA molecule maybe a linear DNA molecule with covalently closed ends having the bacterial sequences removed from the vector.

Importantly, there are no distinct size limitations for the DNA vector encoding ATP7B, although optimal forms of the DNA vector composition will preferably be as small as possible to increase delivery efficiency and/or yield from DNA manufacturing. This was demonstrated by delivering 8.4 kb size of plasmid DNA into the livers of mice and pigs by hydrodynamic gene delivery, respectively.

The promoter sequence is a crucial aspect to designing any gene therapy. As mentioned above, AAV gene therapy has significant size limitations, which has focused the AAV gene therapy field on small, short promoters that are expected to have high expression activity. Many smaller promoters are largely unregulated with respect to the gene being encoded, lacking the native gene-specific regulation of many genes to sense extracellular and intracellular conditions.

For the purposes of ATP7B gene therapy, an unexpected and surprising result of approaches that leverages high-level, unregulated expression has been noted. When a plasmid encoding a liver-specific promoter based on a chimeric enhancer-promoter combination of APO HCR and alpha-1 antitrypsin (AAT) promoter was delivered, the plasmid leads to efficient expression of ATP7B, although this expression was found to be toxic to the hepatocytes in rodents when vector copies are delivered into cells, resulting in the loss of ATP7B expression. Such overexpression toxicity has never been reported in the literature and is therefore very surprising.

However, upon testing these same constructs in pig models that are similar in size to humans, no overexpression toxicity was observed. This points to a previously unreported observation that expression of transgenes may be lower in larger animals, such that the overexpression toxicity is less of a concern in larger animals as the DNA doses injected. There is the potential for higher DNA dose ranges (ex: >50 mg pDNA) causing overexpression toxicity like in rodents. In that case, the disclosure endorses keeping such pDNA doses below this threshold.

Alternatives could also be considered to address this limitation. As mentioned above, the first solution is that the method of gene therapy can be adjusted, wherein lower concentrations of plasmid DNA encoding ATP7B under the direction of a high level liver-specific promoter is performed. This would serve to titrate the DNA dose and subsequently the amount of ATP7B expression inside the cell. This strategy does have limitations however, since it may lead to fewer cells receiving the ATP7B gene. Moreover, the amount of plasmid DNA distributed among all liver cells is heterogeneous, such that one cannot entirely rule out certain cells receiving too much ATP7B gene and thus too much ATP7B expression.

As a second solution, ATP7B could be expressed from promoters that are regulated by the amount of copper and/or metals in the environment. Examples of these promoters include the native ATP7B promoter itself, as well as the metallothionein promoter. In addition, hybrid promoters are contemplated such that the core promoter is a ubiquitous liver-specific promoter such as alpha-one antitrypsin (AAT) promoter, but the enhancer element that upregulates promoter expression will be based on a metal responsive element (MRE) found in the ATP7B or metallothionein promoters. Concerning core promoters that are regulated by copper levels themselves such as ATP7B, various different combinations are contemplated. In some embodiments the native ATP7B promoter without any sequence alterations will be utilized up to 1500, 1200 or 1000 bp's ahead of the native ATP7B will be utilized. Since the exact ATP7B promoter is undefined, and multiple different sequence lengths as listed could also be utilized. In preferred embodiments of the disclosure, at least 500 base pairs of sequence ahead of the native ATG start codon in the human genome of ATP7B utilized. In another preferred embodiment, at least 1000 base pairs upstream of the ATG sequence in the human genome of ATP7B. In other embodiments, a synthetic promoter with multiple repeats of MRE elements will be utilized to drive ATP7B expression.

In certain embodiments, a core region of the ATP7B promoter will be utilized, which encodes sequences containing only the positive regulatory elements of ATP7B promoter (−800 bp ahead of the native start codon of ATP7B gene) resulting in higher expression. In other embodiments, the negative regulatory regions of the ATP7B promoter will be utilized be kept in.

Several additional modifications to the native ATP7B promoter beyond using different sequence lengths of the native ATP7B promoter are envisioned. These primarily focus on the addition of enhancer elements to the ATP7B promoter to augment gene expression while maintaining regulation. While ATP7B promoters have the benefit of maintaining natural regulatory process to sense copper levels, the limitation is that the transcript level is relatively low compared to other liver-specific promoters used in gene therapy. This is particularly important when using episomal plasmid DNA vectors, which have generally lower expression of mRNA compared to the same gene on the host chromosome. The ATP7B promoter is significantly weaker versus the native alpha-1 antitrypsin (AAT) promoter by comparison, which corresponds to the relative amounts of each proteins that are required for normal human physiology. Indeed, the ATP7B native promoter off an episomal DNA is only weakly active in mouse liver compared to the AAT promoter.

To address these limitations, liver-specific enhancer elements such as APO HCR and ApoE enhancer are envisioned to be added to the 5′ end of the ATP7B promoter in certain embodiments of the disclosure. In other embodiments of the disclosure, enhancers derived from different viruses such as simian virus 40 and hepatitis B virus could also be added, which have general properties of increasing promoter strength, while maintaining the specificity of cell-type expression and regulation. Examples of these enhancer elements include SV40 enhancer and HBV enhancer I and HBV enhancer II. In other embodiments of the disclosure, negative regulatory elements could be removed from the ATP7B promoter in the region −1200 to −800 and replaced with liver-specific or viral enhancers to enhance the expression ATP7B.

In still yet other embodiments of the disclosure, a liver specific core promoter could still be utilized, but the enhancer element could be derived from metal regulatory elements (MRE's) that are governed by transcription factors, which sense the concentrations of different metals inside the cell, including copper. A DNA sequence that includes multiple different copies of these metal regulatable elements (MRE's) could be included 5′ to a core liver specific promoter such as alpha-1 antitrypsin, in order to achieve effective enhancement of gene expression in response to the levels of metal inside the cell.

Alternative promoters beyond ATP7B can also be contemplated for the DNA vector. Metallothionein is an important protein that helps sequester different metal ions in the body. The metallothionein promoter also responds to increased levels of metal ions in order to facilitate more production of metallothionein protein to sequester the metal ions. As such, the metallothionein promoter is another alternative to regulate ATP7B expression inside hepatocytes.

Other elements of the DNA vector are also crucial for optimizing expression of the ATP7B gene, particularly in the situation where the promoter is relatively weak and is being regulated by copper status. In these situations, other elements can be utilized to counteract the relatively weaker expression from plasmid DNA in order to achieve sufficiently high ATP7B levels that are still regulated by copper levels. In one embodiment of the disclosure, the 5′ UTR will have an intron introduced into it, in order to increase mRNA export from the nucleus an ultimately expression in the cytoplasm. These introns will be non-native to the 5′ UTR of human ATP7B, as well as the metallothionein or alpha-1 antitrypsin promoters contemplated. In optimal embodiments, it would include miniature intronic elements from certain viral sequences such as SV40 or MVM.

In other embodiments, particularly when the native ATP7B promoter is utilized, the native ATP7B 5′ UTR will be utilized in the vector, such that introns will be introduced into the coding sequence of the ATP7B protein, where they otherwise do not naturally exist. In these embodiments, at least one intron will be introduced, although other embodiments will employ the use of two or more introns.

In other embodiments of the disclosure, a 3′ UTR would be added downstream of the ATP7B coding sequence that would lead to the enhancement of expression. The 3′ UTR in these embodiments which function in order to increase the half-life of mRNA inside the cell's cytoplasm, as well as by enhancing translational potency of a given mRNA molecule. This would effectively increase ATP7B expression without interrupting its ultimate regulation from its promoter element. Examples of 3′ UTR elements that can be used for this purpose include the human alpha and beta globin gene 3′ UTR regions.

A disadvantage of AAV vectors and plasmid DNA vectors is their episomal status. Because the DNA vectors are not integrated into the genome, they could be lost overtime with the division of hepatocytes. In certain embodiments of the disclosure, sequences could be added to the DNA vector that would allow for replication during mitosis in cells. These sequences would be derived from scaffold matrix attachment regions (S/MAR) elements, which could be included in the DNA vector to facilitate replication. In some embodiments, S/MAR elements would optimally be placed 3′ region to or 3′ UTR of the ATP7B gene cassette. The S/MAR sequence on the vector will facilitate effective replication of the episomal DNA vector in the disclosure with hepatocyte mitosis, such that hepatocytes expressing ATP7B would be protected from copper toxicity, and thus the percentage of positive hepatocytes will increase within the liver over time.

Another element of the vector would be a DNA sequence directing polyadenylation of all the mRNA transcripts generated, as is customary in most expression vectors. Among the examples of polyadenylation sequences that could be included in this vector include SV40 polyadenylation sequence, human growth hormone polyadenylation sequence, bovine growth hormone polyadenylation sequence, and rabbit beta-globin polyadenylation sequence.

The coding sequence, itself, of ATP7B is a key feature of the vector, which can improve the vector potency and resultant therapeutic activity. In optimal embodiments of the disclosure, the full length ATP7B gene will be utilized. The coding sequence may be interrupted with additional introns in order to increase expression, but every native amino acid to ATP7B will be coded for. In certain embodiments of the disclosure, a small c-terminal protein tag may be added in order to distinguish between endogenous mutant ATP7B and the vector delivered wild-type ATP7B transgene. This C terminal tag in preferred embodiments should have no disruption of native ATP7B function and/or trafficking within the cell. Examples of c-terminal tags that could be utilized include the C9-tag derived from the c-terminal 9 amino acids from the human rhodopsin protein, and the c-myc tag. The exact DNA sequence of ATP7B may be the same as that encoded in the human genome. In other preferred embodiments, the DNA coding sequence is codon optimize to increase the levels of usage of common DNA codons in the human cells, such that the overall protein expression is increased. In these embodiments, the DNA sequence will be completely different from the native human ATP7B sequence, but the protein coded will be the same wild-type ATP7B gene. In even more preferred embodiments, the coding sequence of ATP7B preferentially uses codons that are utilized in the liver at high levels.

In preferred embodiments of the disclosure, different small nucleotide polymorphisms (SNPs) will be incorporated into the coding sequence of ATP7B. ATP7B is an enormous protein (1,465 amino acids), and as such that there exists no canonical truly wild-type sequence. Different SNP's exist in the human population, and it is not obvious which of them to utilize and in which combinations. Depending on the combination of SNP's utilized, there will be different levels of ATP7B activity in its copper transporting properties. In preferred embodiments of the disclosure, SNP's existing at K832 and R952 will be incorporated to increase the copper transport activity of ATP7B. This will lead to higher overall reduction in copper levels in spite of lower amounts of ATP7B protein. Search use of hyperactive variants of ATP7B for use in gene therapy has not been previously reported in the literature. This strategy is analogous to the use of hyperactive human FIX variants in the treatment of hemophilia B.

The DNA vector is optimally delivered through hydrodynamic injection. Routes of hydrodynamic injection include vascular or biliary routes. In either route, the DNA vector would be dissolved in a pharmaceutically acceptable solution, such as normal saline, phosphate buffered solution, lactate ringer's solution, or dextrose solution. Optimal pressure will be obtained that creates pores in cell membranes in order to deliver the DNA vector inside cells. In other embodiments, the DNA vector could be encapsulated within a lipid particle or a lipid nanoparticle to facilitate DNA protection and cell uptake. The compositions of the lipids in these particles may vary.

In particular, hydrodynamic delivery through biliary routes offers higher efficiency of hepatocyte transfection compared to vascular routes, while also serving to allow for gene delivery into cholangiocytes. ATP7B natively expresses in hepatocytes and cholangiocytes. Other gene therapy approaches with AAV only serve to target hepatocytes currently. It is reasonable that also targeting expression into cholangiocytes would better allow for enhanced copper excretion, which naturally occurs through the biliary tract. Therefore, the current disclosure is unique in targeting both cell types for enhanced gene therapy for Wilson's Disease.

### EXAMPLES

### Example 1: Validation of Plasmid DNA Vector Encoding Human ATP7B,C9 In Vitro

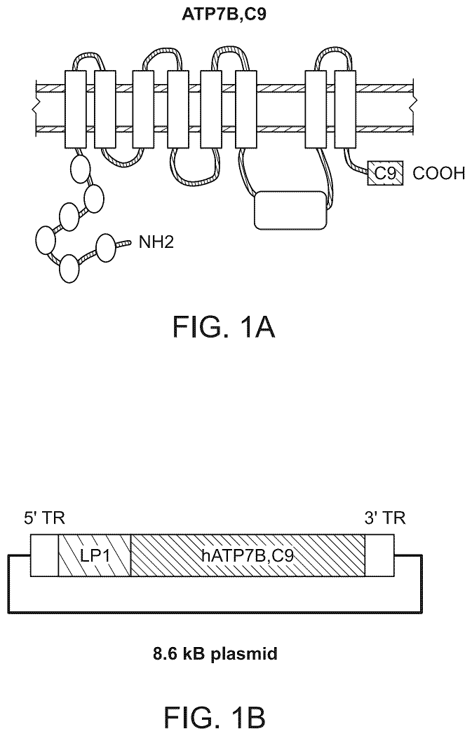

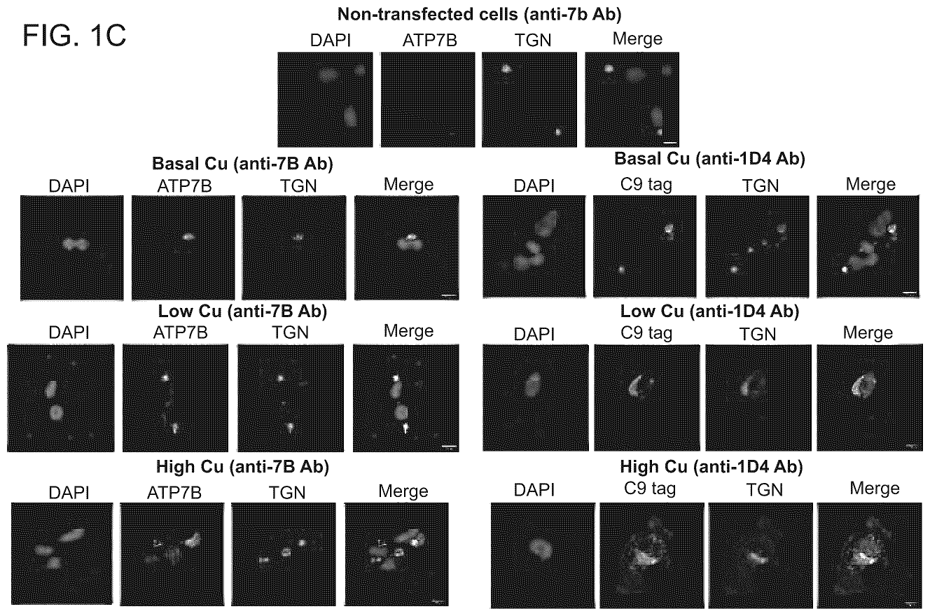

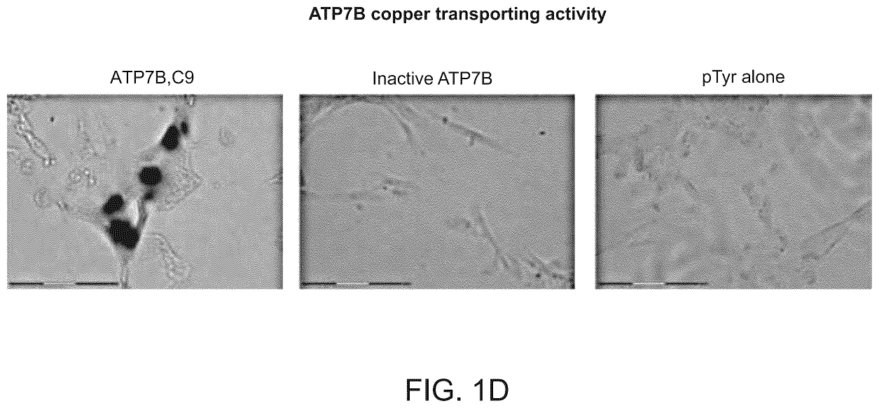

A pDNA vector to efficiently express hATP7B after transfection into cells was constructed. To facilitate detection of translated ATP7B protein in wild-type pigs, a C-terminal tag encoding the last nine amino acids from the human rhodopsin protein, (e.g., C9-tag; FIG. 1A) was added to ATP7B. The C9-tag may be located at the protein's C-terminus and has commonly been used to tag membrane proteins. However, the C9-tag may also be used to tag an epitope that is located intracellularly (Molday et al 2014). As shown in FIG. 1B, the hATP7B,C9 gene was cloned into a plasmid DNA vector driven by a liver-specific promoter, with expression cassette located within piggyBac terminal repeats to mediate integration.

While a C-terminal myc tag on ATP7B has been employed without affecting ATP7B function (Zhu et al 2013), the experiments herein sought to validate that introduced C9-tag would not alter ATP7B localization and function. ATP7B,C9 was first transfected into YST cells, which lack native ATP7B expression. Immunofluorescent staining demonstrated that ATP7B,C9 can be efficiently detected within anti-C9 antibody, with the staining pattern was indistinguishable from ATP7B (FIG. 1C). Importantly, this staining co-localized with the trans-golgi network (TGN) at basal copper levels, indicating proper ATP7B,C9 localization. ATP7B,C9 was then assessed to determine if it retained proper movement inside cells in response to copper. In high copper conditions it was observed that ATP7B,C9 moved out in speckled pattern beyond the TGN in the expected pattern for wildtype ATP7B (FIG. 1C). In low copper conditions, ATP7B,C9 remained in the TGN as expected.

Functional assays with ATP7B,C9 were then performed. Copper transport into the secretory pathway by ATP7B,C9 was monitored by evaluating copper loading into the copper-dependent enzyme tyrosinase (Roy et al 2020). Formation of eumelanin pigment by tyrosinase can be visually appreciated, indicating successful. copper transport. Efficient production of eumelanin was observed with ATP7B,C9, while an inactive mutant ATP7B lacked any eumelanin, along with transfected cells without any ATP7B. (FIG. 1D).

### Example 2: Validation Function of ATP7B after Hydrodynamic Injection in a Mouse Model of Wilson's Disease

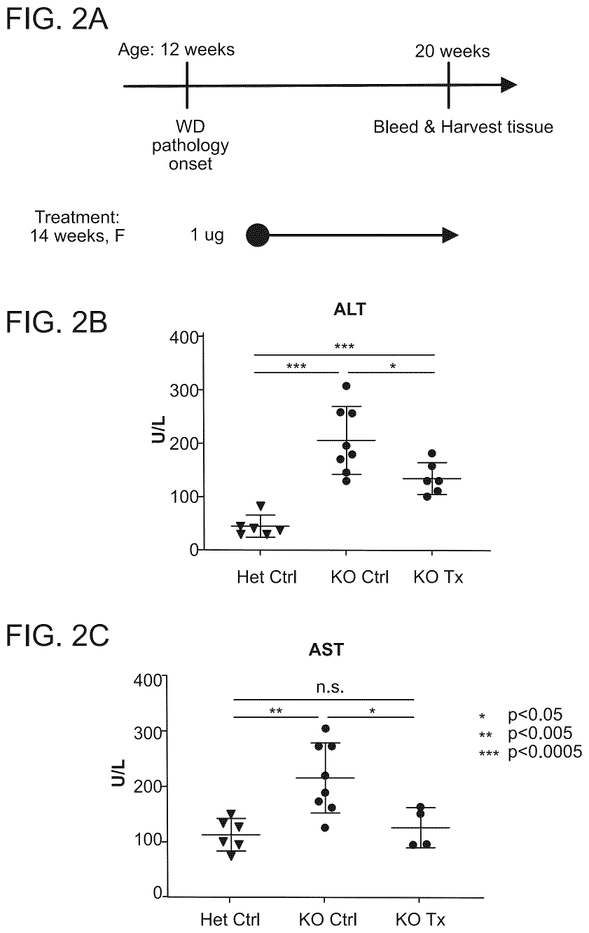

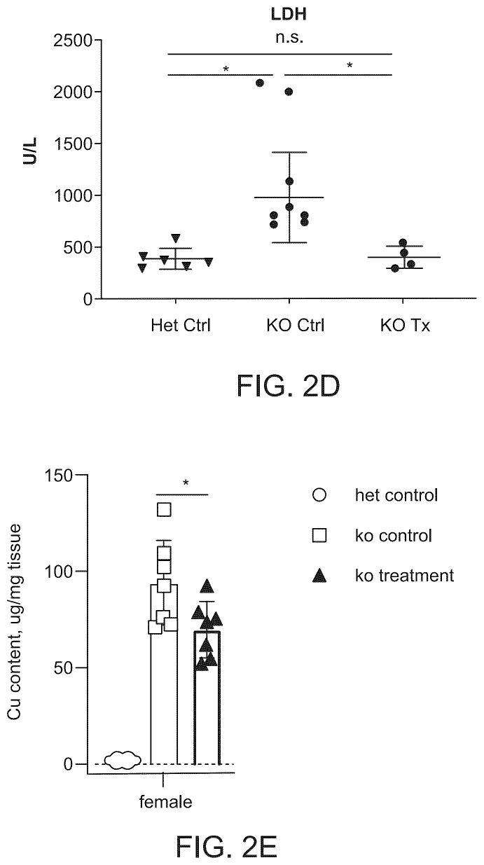

The techniques herein sought next to validate the function of ATP7B,C9 after non-viral delivery of pDNA into WD mice. WD mice with pathology already present were treated, which starts around 12 weeks age in C57BL6 mice. This would show that gene delivery a WD liver with injury was possible, important seen most WD patients already have liver phenotype at diagnosis. The techniques herein chose to employ a low dose of pDNA, 1 μg, was employed to see the impact of fewer hepatocytes transfected, and whether these transfected cells would expand over time with integrated hATP7B,C9. Mice were injected around 14 weeks ago, and evaluated at 20 weeks (FIG. 2A), a timepoint used for analysis in a previous study (Muchenditsi et al). After only 6 weeks of therapy, several markers in the Wilson's disease treated mice could be significantly reduced, including ALT, AST, and LDH (FIGS. 2B-D). Other markers (alkaline phosphatase, total bilirubin) did not show any significant difference from the untreated group (FIG. 5). Hepatic copper levels were also reduced by 27% in treated WD mice, indicating successful copper transport function of ATP7B,C9 in vivo (FIG. 2E).

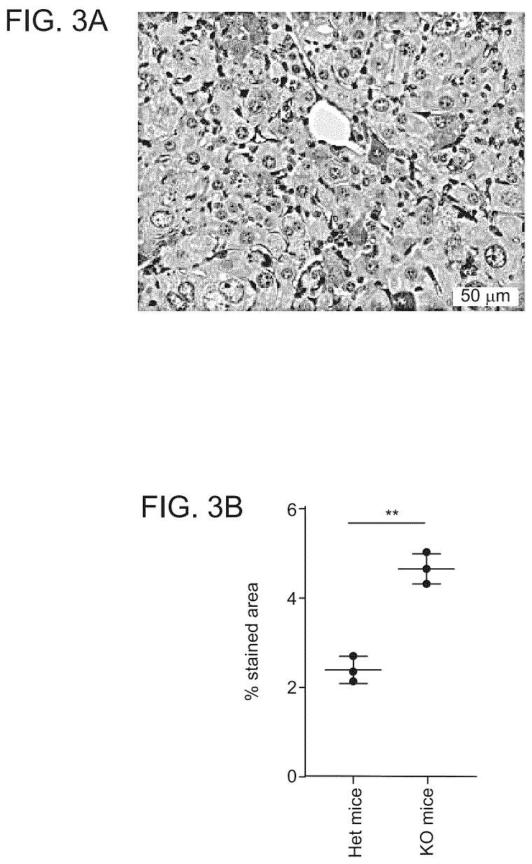

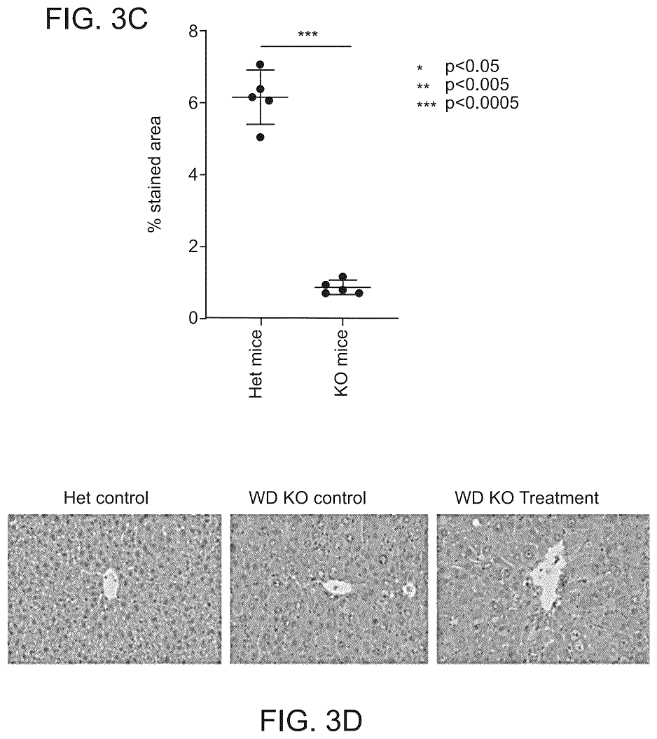

Looking at gene delivery into the liver, the presence of ATP7B,C9 hepatocytes was observed by immunohistochemistry for the C9-tag in the mice harvested at 20 weeks, confirming their presence to mediate the copper reduction (FIG. 3A). Hepatocytes exhibited a range of staining intensities, likely reflecting different expression. The percentage of ATP7B,C9 hepatocytes was calculated to be 4.65% area stained of hepatocytes in WD mice injected with 1 μg of plasmid DNA (FIG. 3B). This percentage was compared to heterozygous mice injected with ATP7B,C9, finding significantly less gene delivery had occurred with only 2.40% stained hepatocyte area in Het mice (p=0.0009) (FIG. 3B). To determine whether this difference could be due to the efficiency of the initial delivery in a WD mice with altered liver architecture a reporter GFP plasmid was used, and it was found that the stained area of GFP-positive cells was significantly less in WD mice (0.85%) versus Het mice (6.11%), pointing to an inhibition of HTVI delivery by diseased WD liver (p<0.0001) (FIG. 3C). The overall histology among the mice did not demonstrate noticeable differences from treated to untreated mice (FIG. 3D), which matches the transaminase elevation still present in treated groups.

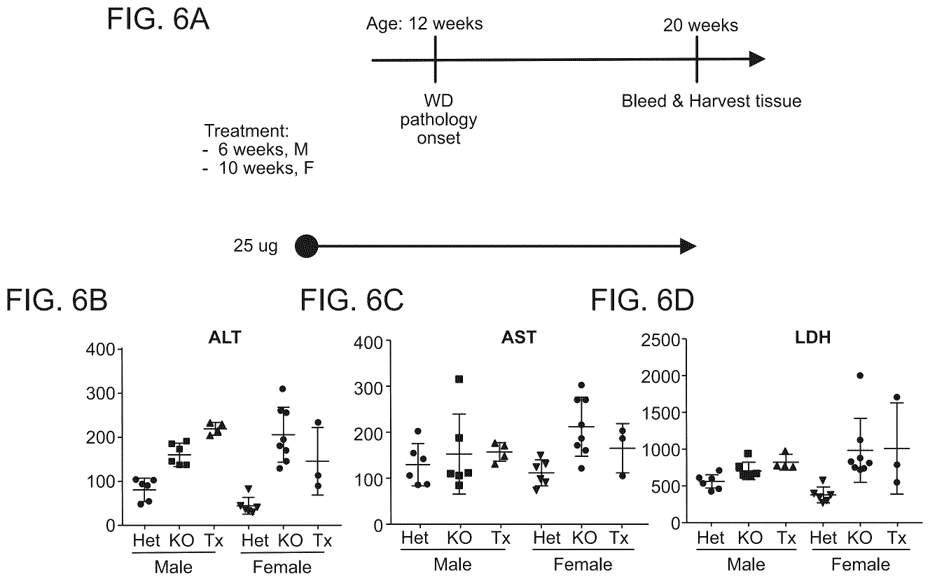

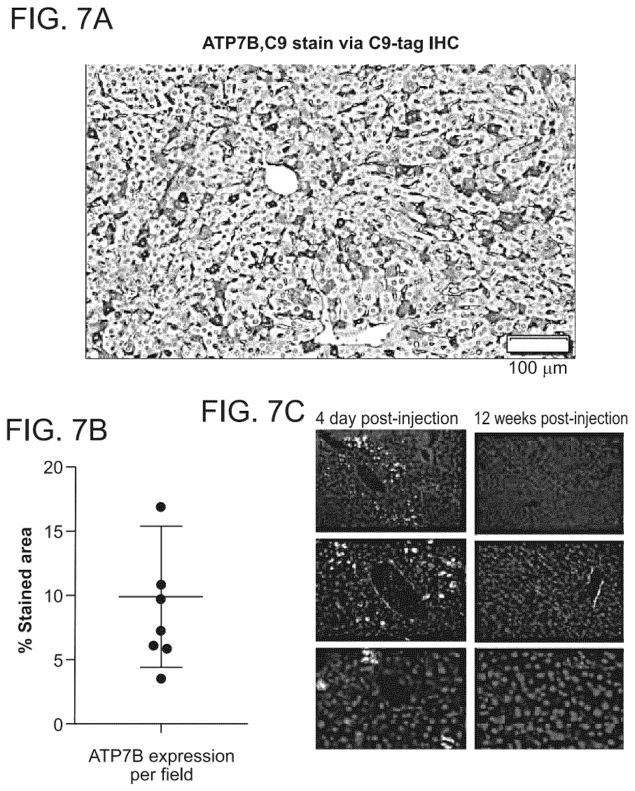

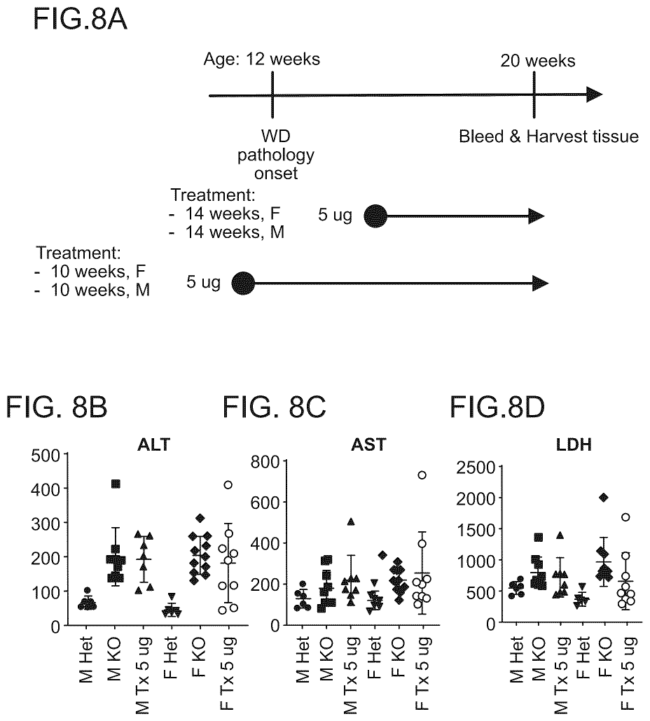

The ability of higher doses of pDNA hydrodynamic injection to increase transfection percentage was next evaluated. WD mice treated with 25 μg pT-LP1-hATP7B,C9 surprisingly did not exhibit improvements in liver enzymes (FIG. 6), which correlated with the absence of ATP7B,C9 protein on immunofluorescent staining (FIG. 7). This contrasts with 9.86% of hepatocytes transfected 4 days post-injection with 25 μg pDNA (FIG. 7). A reduced dose of 5 μg pT-LP1-hATP7B,C9 had similar results, with lack of improvement in liver transaminases (FIG. 8).

### Example 3: Biliary Hydrodynamic Injection of ATP7B Plasmid in Pigs

To assess the ability of hydrodynamic injection to treat human WD patients, hydrodynamic injection of hATP7B,C9 pDNA into a human-sized animal model was tested. The systemic pressure increase from vascular hydrodynamic tail vein injection in mice is not applicable to human patients. The techniques herein have pioneered a strategy of hydrodynamic injection through the biliary system into pigs, which efficiently branches into all lobes and contacts all hepatocytes (Kumbhari et al 2018; Huang et al 2021). In addition, the techniques herein have demonstrated that biliary hydrodynamic injection in pigs could actually achieve a higher transfection efficiency then mouse tail vein injection (Kruse et al 2021). Because of this, further experiments to continue optimizing the pDNA dose were not conducted in mice (FIGS. 6-8), as the data ultimately is not clinically translatable to human patients, and has lower, limited gene transfection efficiency.

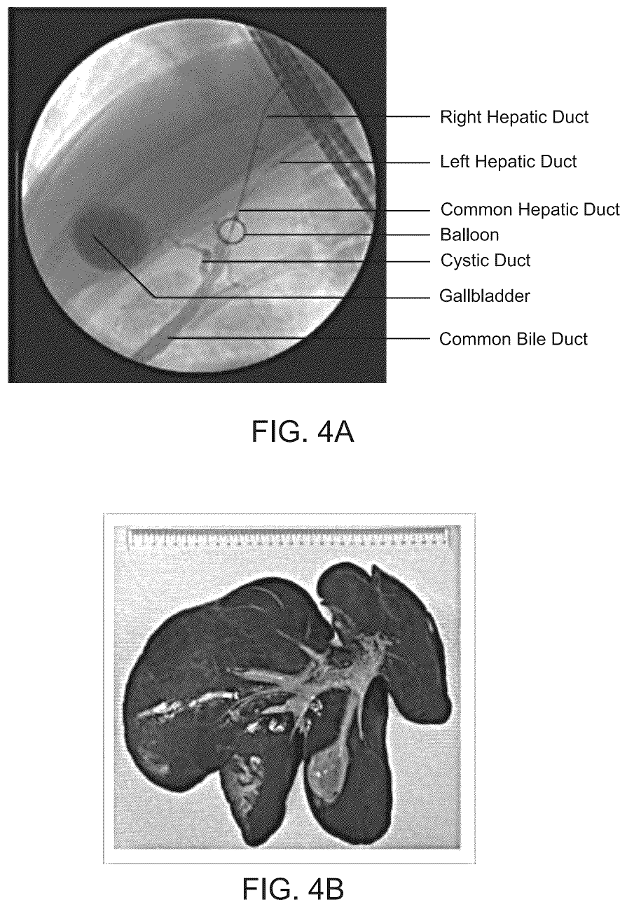

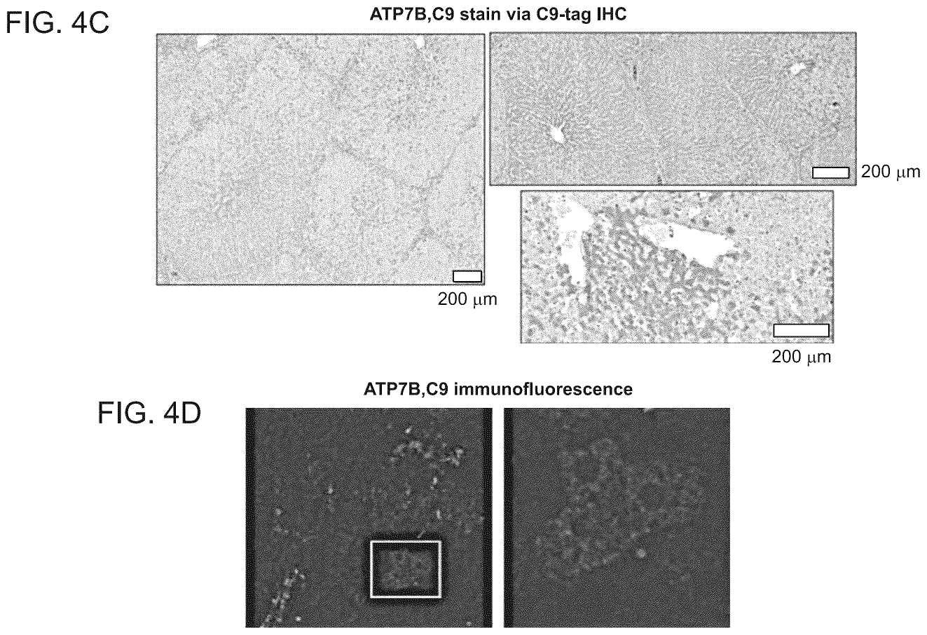

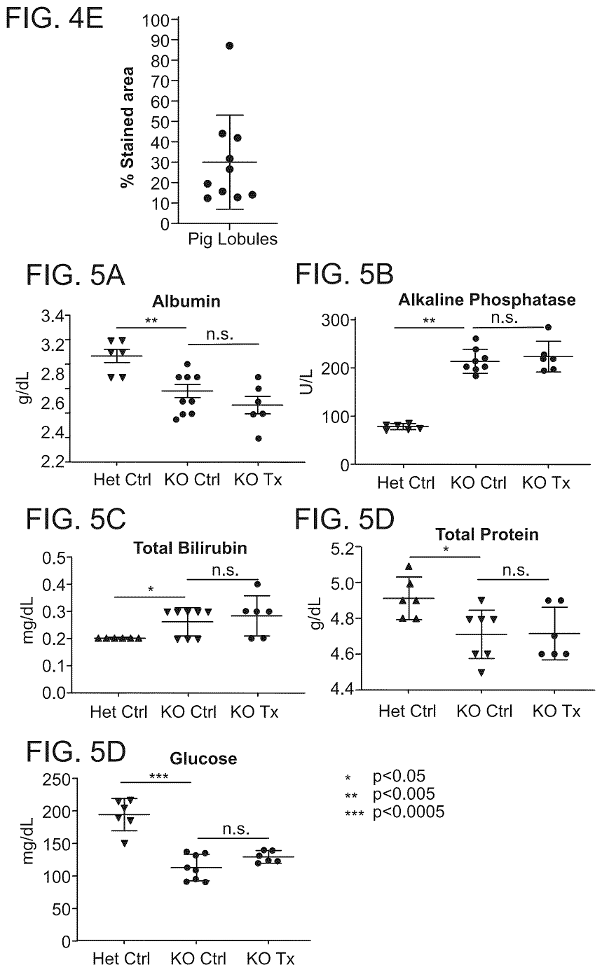

Two pigs weighing between 40-50 kg were obtained for testing hATP7B,C9 gene delivery via endoscopic retrograde cholangio-pancreatography (ERCP). Pigs were monitored before and after injection and demonstrated no elevation in liver transaminases (data not shown). Fluoroscopy before the injection confirmed catheter placement of the catheter in the common hepatic duct (FIG. 4A). 10 mg of ATP7B,C9 pDNA was injected into the first pig. As shown in FIG. 4B, pig liver was harvested at day 1 post-injection demonstrating no abnormalities or lesions after biliary hydrodynamic injection. DNA was extracted from all lobes, and PCR testing was able to correctly localize ATP7B DNA in all of liver lobes including proximal and distal locations in the lobe compared to the injection site (data not shown). Evaluating for protein expression by immunohistochemistry (IHC), ATP7B,C9 could be detected in all pig liver lobes. As shown in FIG. 4C, there were positive ATP7B,C9 staining along the lobular borders as well as near the central vein across all three zones. Immunostained cells were clearly distinguishable from neighboring negative hepatocytes, with some variation in intensity of staining. Interestingly, certain lobules appeared to express ATP7B,C9 among almost 80-90% of hepatocytes (FIG. 4C). On total, the average area of ATP7B-positive hepatocytes was 30.58% in a pig transfected with 10 mg of pDNA (FIG. 4E). Other pigs injected demonstrated similar efficacy.

Finally, the localization of ATP7B,C9 protein in pig hepatocytes was confirmed by immunofluorescence staining (FIG. 4D). ATP7B,C9 was observed to be correctly located in pig hepatocytes, with a pattern distinguishing from endogenous ATP7B in pigs, as well as ATP7B in mouse hepatocytes.

### Conclusions

In this study, the successful gene delivery of hATP7B into a large animal model has been demonstrated for the first time in the literature. The techniques herein validated that hATP7B expressed from pDNA is functional in vitro and in WD mouse models, before achieving significant transfection of pig hepatocytes with hATP7B,C9. Given that the pigs employed in this study are approximately the size of an adult human female and that the techniques herein employed a routine clinical procedure in ERCP, it is believed that this approach is translatable to WD patients. This study is an important step in demonstrating the potential of gene therapy for WD.

It has been demonstrated hATP7B can be expressed from a hydrodynamically delivered plasmid vector in mice and pigs for the first time. Non-viral hydrodynamic gene delivery of pDNA has no defined size limit for the DNA vector, in comparison to AAV vectors previously employed. The successful expression of an 8.6 kB pDNA in pigs, which is ~80% larger than the AAV genome packaging limit, has been achieved. Without being bound by theory, it is believed that the techniques herein suggest that the use of full-length hATP7B may be preferable to the use of truncated miniature ATP7B, which lack several metal binding domains, and have been used in AAV studies (Leng et al 2019).

Crucially, hydrodynamic gene delivery of hATP7B DNA could occur in a diseased WD liver yielding functional protein in WD mice, reducing liver injury and decreasing hepatic copper content. The level of hATP7B hepatocyte transfection (4.6%) was not enough to completely cure WD disease in mice, however. This is consistent with previous WD gene therapy studies, which suggested a level of 20% is necessary for cure in a WD mouse model (Murillo et al 2019). Increasing pDNA doses in mice mildly increased transfection efficiency to 10%, but this appeared to result in overexpression toxicity or immune responses to ATP7B since mice tested at 2 months post-injection were negative for ATP7B,C9 staining. As a comparison, hydrodynamic delivery of low density lipoprotein receptor (LDLR) DNA in the treatment of familial hypercholesterolemia in mouse models has yielded toxicity from overexpression with too much lipid accumulation (Cichon et al 2004). As a membrane protein, ATP7B could accumulate inside the cell's TGN eventually disrupting other cell processes. Importantly, a small dose of 1 μg of ATP7B in mice was able to achieve therapeutic benefits despite transfecting very few cells (<4.6%). It may be important to further study immune responses against or overexpression of ATP7B in any Wilson's disease gene therapy trial whether from an AAV vector or pDNA.

An unresolved question in gene therapy for WD is if the corrected hepatocytes have a proliferative advantage compared to the surrounding diseased hepatocytes. Proliferative advantages are notably observed in two other monogenetic liver diseases, hereditary tyrosinemia type I (Hickey et al 2016) and alpha-1 antitrypsin disease (Borel et al 2017). In these disorders, an initial population of a few percent corrected cells can expand over months to greater than half the liver population. WD could have the same mechanism, given the hepatocyte damage, given ongoing hepatocyte damage. The techniques herein showed that the initial gene delivery of reporter GFP by HTVI was profoundly inhibited by the distorted architecture of the WD liver. Surprisingly, WD mice injected with ATP7B 6-8 weeks later actually showed a higher percentage area of ATP7B compared to Het mice. Together, this suggests robust expansion of ATP7B-positive hepatocytes can occur after expression of ATP7B by a transposon system, estimated at 14-fold expansion over 6-8 weeks.

This result is surprising since previous studies with hepatocyte cell therapies for WD failed to show significant competitive advantage for hepatocytes in WD models without additional augmentation. One study of hepatocyte cell therapy in WD rats observed hepatocyte repopulation over 1 year to almost 100% of cells (Irani et al 2001), but that investigation used chemicals (retrosine) to artificially augment liver damage to increase the advantage. A more recent cell therapy studied required resection of 30% of the liver to help drive ATP7B+ hepatocyte repopulation (Cai et al 2022). In a more similar gene therapy study, lentiviral transduction of ATP7B in gestational mice did not note expansion, although the study was able to ameliorate biomarkers of the disease (Roybal et al 2012). The differences between the present study and the previous reports may be the higher expression mediated by non-viral gene delivery of ATP7B, yielding more competitive hepatocytes, as well as the even distribution of ATP7B cells across the liver tissue, as opposed to cell therapy efforts.

Promisingly, the techniques herein were able to achieve greater than 20% of hepatocytes expressing hATP7B after gene delivery into pigs. A hepatocyte cell therapy in Long-Evans cinnamon rat model of WD suggested complete correction with 20% of ATP7B-positive hepatocytes (Irani et al 2001). A recent gene therapy study suggested correction above 20% is sufficient to normalize phenotype, with significant improvement in copper reduction observed above 10% (Murillo et al 2019). These two studies suggest that a small number of ATP7B-positive hepatocytes can act as a sink for copper to compensate for the entire organ. The present data fall in line with these previous studies, wherein a 4.6% transfection rate of ATP7B-positive hepatocytes in WD mice yielded a 27% copper reduction, stoichiometric with the 20% threshold that has been published. While it is possible that biliary hydrodynamic delivery efficiency could drop in WD liver, similar to what occurred via HTVI, any shortfall in patients should be overcome with additional expansion of ATP7B-expressing hepatocytes. Together, the techniques herein show that delivery achievement in pigs may be enough to potential fully reverse disease pathology in WD patients.

In comparing the present studies to AAV gene therapy approaches for WD, AAV can be dosed to transduce the majority of hepatocytes in mice, while HTVI is limited only transfected at most 20% of hepatocytes, and in many studies 5-10%. For another difference, ssDNA to dsDNA conversion among AAV genomes is inefficient, however, leading to lower levels of hepatocytes actually expressing hATP7B, while hydrodynamic injection delivers pDNA that is expressed within hours after injection. This may have resulted in lower net levels of ATP7B expressed across more hepatocytes for AAV approaches, contrasting with the present study. Moreover, previous AAV gene therapy papers lacked enhancer elements in the liver-specific promoter in order to make expression cassette fit in the AAV backbone, further reducing expression, while the present study included these elements to increase ATP7B expression within the cell (Murillo et al 2016; Murillo et al 2019). It will be interesting to see how potential AAV trials progress for WD, given that AAV generally becomes less efficient when scaling from mice to human patients.

This study had several limitations. It was limited in the length of time mice were treated for WD. The techniques herein demonstrated the functionality of hATP7B gene in mice after delivery, though they did not seek to optimize treatment of mice for cure for WD, since the delivery routes and efficiencies are markedly different between mice and pigs. The study is also limited by the length of time, one day, during which ATP7B expression was analyzed in pigs. While the focus was on defining the relative transfection efficiency, future studies will explore the duration of ATP7B expression in pigs.

In conclusion, the techniques herein demonstrate gene delivery of hATP7B DNA into a human-sized animal model for the first time. It was demonstrated that the plasmid DNA-mediated expression of ATP7B creates functional protein in both tissue and mouse models as well. Given that the techniques herein use clinically available equipment and an ERCP procedure in routine clinical practice today, the present disclosure indicates that the technique may be readily translated into the treatment of Wilson's disease patients.

The non-viral approach disclosed herein is appealing for the potential to redose gene therapy over time, improves the safety of the system, and represents significant cost savings in manufacturing. Future studies will continue to determine the optimal dose of ATP7B,C9 plasmid to utilize, as well as define the duration and stability of ATP7B expression in pigs, paving the way for potential clinical trials.

### Example 4: Methods and Materials

### Plasmid Construction

A human ATP7B (hATP7B) cDNA previously studied by the Lutsenko Lab was utilized that contained polymorphisms at location K832 and R952, which were found in a previous study to mediate increase copper transporting activity (McCann et al 2019). The carboxy-terminus tag corresponding to the 9 terminal acid amino residues of the bovine rhodopsin gene (TETSQVAPA) was added by PCR cloning onto the hATP7B gene. The hATP7B,C9 gene was inserted into the pT-LP1-hFIX vector developed by the Kumbhari lab, removing the hFIX gene via XbaI and BglII restriction sites. This vector was previous constructed through gene synthesis (Bio Basic). The LP1 promoter is derived from a composite of the human apolipoprotein hepatic control region and the human alpha-1-antitrypsin (hAAT) gene promoter (Nathwani et al 2006), while the entire expression cassette is located between piggyBac transposon terminal repeats to facilitate integration (Wilson et al 2007). The plasmid, pCMV-hyPBase, was previously synthesized by the Kumbhari lab encoding a hyperactive piggyBac transposase to facilitate gene integration (Doherty et al 2007). DNA was prepared for injection using QIAgen Plasmid Maxi prep kit for mouse injections and ZymoPURE™ II Plasmid Gigaprep Kit (Zymo Research) for pig injections.

### In Vitro Copper Loading Assay

Menkes disease fibroblast (YST) cells, lacking active ATP7A and ATP7B, were seeded in 8-well chamber slides at a density of 0.01×106 cells per well. The next day, cells were co-transfected with 100 ng each of either pTyr plasmid alone or with either wt ATP7B plasmid or D1027A GFP-ATP7B (inactive mutant). 20 h after transfection, expression of ATP7B was confirmed by GFP signal in DA samples. The cells were then fixed and incubated with L-DOPA for 3 h, then mounted and imaged by phase contrast microscopy to detect formation of eumelanin pigment. Eumelanin pigment formation indicates successful transfer of copper to tyrosinase, which is otherwise depending on copper for enzymatic function.

### In Vitro ATP7B Movement Assays

YST cells were seeded at a density of 8×103 cells per well in 8-well chamber slides in complete media (CM: DMEM, 10% FBS, 1% penicillin/streptomycin). After 48 h, the cells were transfected with 200 ng of C9-ATP7B plasmid using Lipofectamine LTX and Plus reagent system. After 20 h, the media was changed to either basal (CM with 9 mg/ml cycloheximide), high Cu (basal+100 uM Cu), or low Cu (basal+25 uM TTM) media and incubated for 3 h at 37 °C. The cells were washed with PBS, fixed using 4% PFA for 15 min, permeabilized with 0.1% Triton X-100 for 15 min, and blocked with 5% BSA for 40 min at RT. After washing with PBS, the cells were incubated with primary antibodies in PBS with 0.1% Triton X-100 (mouse 1D4 at 1:400 or rabbit anti-7B at 1:400, sheep TGN46 at 1:600) for 1 h at RT. The cells were washed twice with PBST for 5 min and once with PBS for 5 min. The cells were then incubated with secondary antibodies in PBS with 0.1% Triton X-100 (anti-mouse Alexa488 at 1:400 or anti-rabbit Alexa 488 at 1:1000, anti-sheep Alexa 555 at 1:1000) for 1 h at RT, protected from light. The cells were washed twice with PBST for 5 min and once with PBS for 5 min, dried, mounted using Fluoromount-DAPI, and cured in the dark. The cells were imaged using LSM 800 confocal microscope with 63× oil lens.

### Mouse Experiments

All mouse studies were conducted under an approved protocol #MO17M385 by the Johns Hopkins IACUC committee. A C57BL6 mouse model with a null ATP7B mutation was previously developed by the Lutsenko Lab and utilized in all experiments (Muchenditsi et al 2021). Mouse were bred with homozygous AT7B KO males crossed with ATP7B heterozygous females. This led to the generation of heterozygous and homozygous ATP7B littermates that were utilized in all experiments.

Mice were injected under established protocols for hydrodynamic tail vein injection (Liu et al 1999). Briefly, mice were warmed under heat lamp to induce vasodilation of their lateral tail veins. Using a 27 gauge needle, pDNA in saline solution corresponding to 10% of the body weight was injected into the mice within 5-7 seconds. Different amounts of pT-LP1-ATP7B,C9 and pCMV-hyPBase were injected depending on experiment. In the low-dose cohort, 1 μg pT-LP1-ATP7B,C9 and 1 μg pCMV-hyPBase were injected. In the middle dose cohort, 5 μg pT-LP1-ATP7B,C9 and 5 μg pCMV-hyPBase were injected. In the high dose cohort, 25 μg pT-LP1-ATP7B,C9 and 10 μg pCMV-hyPBase were injected.

Serum was obtained by retro-orbital bleed from mice, and chemistries analyzed by the Johns Hopkins Phenotyping Core. Serum with noted hemolysis were excluded from analysis for AST and LDH. Mice were euthanized and perfused with saline before liver harvest and tissue analysis.

### Biliary Hydrodynamic Injection

Pig experiments under the approval of the University of Maryland Baltimore IACUC #0720003. A detailed protocol for biliary hydrodynamic injection was previously described (Kumbhari et al 2018; Huang et al 2021). Briefly, all pigs were anesthetized for the procedure and monitored throughout for heart rate, blood pressure, and ventilation. An endoscope was advanced through the mouth and eventually into the small intestine. A catheter was next advanced through the ampulla of Vater into the common bile duct. After further catheter advancement, the balloon on the catheter was subsequently inflated within the common hepatic duct. The catheter was connected to a power injector (Medrad Mark V Arterion), and the pigs were injected with pDNA dissolved in normal saline solution. Injection proceeded at parameters of 40 mL volume at 4 mL/second. Blood draws were collected before and after hydrodynamic injection into pigs to monitor for liver toxicity.

### Immunostaining

For both mouse and pig studies, the use of a C9-tag was leveraged to distinguish delivered hATP7B from host mouse and pig ATP7B. To detect the C9-tag, the 1D4 monoclonal antibody clone (mouse, Santa Cruz, Cat #57432), which reacts with the 9 carboxy-terminal amino acids from the bovine rhodopsin protein (C9-tag), was used. A mouse-on-mouse (MOM) protocol was used to reduce background staining in mouse liver. For immunofluorescent staining of pig tissue, a polyclonal ATP7B antibody (ThermoFisher Scientific) was utilized with FITC labeled secondary antibody, while 1D4 antibody was utilized with the secondary antibody, goat anti-Mouse Alexa 647. For quantification of mouse transfection efficiency from hydrodynamic injection, individual hepatocytes were counted in random fields using ImageJ (with at least 5 fields counted at 20× magnification per mouse). For quantification of pig transfection efficiency, the stained area by immunohistochemistry was calculated within individual hepatic lobules, the latter of which are easily demarcated by fibrous tissue. Quantification proceeded using ImageJ.

### Copper Measurements

Hepatic copper measurements were obtained as previously described by the Lutsenko Lab (Muchenditsi et al 2021).

### Statistics

Unpaired, parametric t-tests were used to determine statistical significance (p<0.05). All statistical analyses were performed using GraphPad Prism 9.0.0 for Mac, GraphPad Software, San Diego, California USA, www.graphpad.com.

### REFERENCES

1. Członkowska A, Litwin T, Dusek P, Ferenci P, Lutsenko S, Medici V, et al. Wilson disease. Nat Rev Dis Primers. 2018; 4:21-20.
2. Masełbas W, Członkowska A, Litwin T, Niewada M. Persistence with treatment for Wilson disease: a retrospective study. BMC Neurol. 2019; 19:278-6.
3. Poujois A, Sobesky R, Meissner W G, Brunet A-S, Broussolle E, Laurencin C, et al. Liver transplantation as a rescue therapy for severe neurologic forms of Wilson disease. Neurology. 2020; 94:e2189-e2202.
4. Murillo O, Luqui D M, Gazquez C, Martinez-Espartosa D, Navarro-Blasco I, Monreal J I, et al. Long-term metabolic correction of Wilson's disease in a murine model by gene therapy. Journal of Hepatology. 2016; 64:419-426.
5. Murillo O, Moreno D, Gazquez C, Barberia M, Cenzano I, Navarro I, et al. Liver Expression of a MiniATP7B Gene Results in Long-Term Restoration of Copper Homeostasis in a Wilson Disease Model in Mice. Hepatology. 2019; 70:108-126.
6. Leng Y, Li P, Zhou L, Xiao L, Liu Y, Zheng Z, et al. Long-Term Correction of Copper Metabolism in Wilson's Disease Mice with AAV8 Vector Delivering Truncated ATP7B. Human Gene Therapy. 2019; 30:1494-1504.
7. Liu F, Song Y, Liu D. Hydrodynamics-based transfection in animals by systemic administration of plasmid DNA. Gene Therapy. 1999; 6:1258-1266.
8. McCann C J, Jayakanthan S, Siotto M, Yang N, Osipova M, Squitti R, et al. Single nucleotide polymorphisms in the human ATP7B gene modify the properties of the ATP7B protein. Metallomics. 2019; 11:1128-1139.
9. Nathwani A C, Gray J T, Ng CYC, Zhou J, Spence Y, Waddington S N, et al. Self-complementary adeno-associated virus vectors containing a novel liver-specific human factor I X expression cassette enable highly efficient transduction of murine and nonhuman primate liver. Blood. 2006; 107:2653-2661.
10. Wilson M H, Coates C J, George A L. PiggyBac transposon-mediated gene transfer in human cells. Mol Ther. 2007; 15:139-145.
11. Doherty J E, Huye L E, Yusa K, Zhou L, Craig N L, Wilson M H. Hyperactive piggyBac gene transfer in human cells and in vivo. Human Gene Therapy. 2012; 23:311-320.
12. Muchenditsi A, Talbot C C, Gottlieb A, Yang H, Kang B, Boronina T, et al. Systemic deletion of Atp7b modifies the hepatocytes' response to copper overload in the mouse models of Wilson disease. Scientific Reports. 2021; 11:5659-16.
13. Kumbhari V, Li L, Piontek K, Ishida M, Fu R, Khalil B, et al. Successful liver-directed gene delivery by ERCP-guided hydrodynamic injection (with videos). Gastrointest. Endosc. 2018; 88:755-763.e5.
14. Huang Y, Kruse R L, Ding H, Itani M I, Morrison J, Wang Z Z, et al. Parameters of biliary hydrodynamic injection during endoscopic retrograde cholangio-pancreatography in pigs for applications in gene delivery. PLoS ONE. 2021; 16:e0249931.
15. Molday L L, Molday R S. 1D4: a versatile epitope tag for the purification and characterization of expressed membrane and soluble proteins. Methods Mol. Biol. 2014; 1177:1-15.
16. Zhu M, Ni W, Dong Y, Wu Z-Y. EGFP tags affect cellular localization of ATP7B mutants. CNS Neurosci Ther. 2013; 19:346-351.
17. Roy S, McCann C J, Ralle M, Ray K, Ray J, Lutsenko S, et al. Analysis of Wilson disease mutations revealed that interactions between different ATP7B mutants modify their properties. Scientific Reports. 2020; 10:13487-15.
18. Kruse R L, Huang Y, Shum T, Bai L, Ding H, Wang Z Z, et al. Endoscopic-mediated, biliary hydrodynamic injection mediating clinically relevant levels of gene delivery in pig liver. Gastrointest. Endosc. 2021; 94:1119-1130.e4.
19. Irani A N, Malhi H, Slehria S, Gorla G R, Volenberg I, Schilsky M L, et al. Correction of liver disease following transplantation of normal rat hepatocytes into Long-Evans Cinnamon rats modeling Wilson's disease. Mol Ther. 2001; 3:302-309.
20. Murillo O, Moreno D, Gazquez C, Barberia M, Cenzano I, Navarro I, et al. Liver Expression of a MiniATP7B Gene Results in Long-Term Restoration of Copper Homeostasis in a Wilson Disease Model in Mice. Hepatology. 2019; 70:108-126.
21. Cichon G, Willnow T, Herwig S, Uckert W, Löser P, Schmidt H H, et al. Non-physiological overexpression of the low density lipoprotein receptor (LDLr) gene in the liver induces pathological intracellular lipid and cholesterol storage. J. Gene Med. 2004; 6:166-175.
22. Hickey R D, Mao S A, Glorioso J, Elgilani F, Amiot B, Chen H, et al. Curative ex vivo liver-directed gene therapy in a pig model of hereditary tyrosinemia type 1. Science Translational Medicine. 2016; 8:349ra99-349ra99.
23. Borel F, Tang Q, Gernoux G, Greer C, Wang Z, Barzel A, et al. Survival Advantage of Both Human Hepatocyte Xenografts and Genome-Edited Hepatocytes for Treatment of α-1 Antitrypsin Deficiency. Mol Ther. 2017; 25:2477-2489.
24. Cai H, Cheng X, Wang X-P. ATP7B gene therapy of autologous reprogrammed hepatocytes alleviates copper accumulation in a mouse model of Wilson's disease. Hepatology. 2022;
25. Roybal J L, Endo M, Radu A, Gray L, Todorow C A, Zoltick P W, et al. Early gestational gene transfer with targeted ATP7B expression in the liver improves phenotype in a murine model of Wilson's disease. Gene Therapy. 2012; 19:1085-1094.

1. A method of treating a human subject having Wilson's Disease, comprising administering a non-viral DNA vector into liver cells by hydrodynamic injection, wherein a therapeutically effective dose of the non-viral DNA vector is administered to the human subject via the biliary system into a liver of the human subject.

2. The method of claim 1, wherein the administration step occurs via an endoscopic retrograde cholangio-pancreatography procedure; or
wherein the non-viral DNA vector achieves expression in at least 20% of hepatocytes in the human subject after delivery, as detectable by protein or RNA staining; or
wherein the non-viral DNA vector dose is at least about 1 mg, 2 mg, 3 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg of DNA per kilogram of liver weight of the human subject; or
wherein the subject exhibits normalization of their liver copper content and restoration of ceruloplasmin levels; or
wherein the human subject is administered with a copper chelation or zinc therapy prior to administration of the non-viral DNA vector; or
wherein the hepatic pathology of the human subject is normalized and disruption of the efficiency of biliary hydrodynamic gene delivery is avoided; or
wherein the copper chelation therapy is selected for D-penicillamine (DPA) or trientine (TETA); or
wherein the human subject possesses normal ALT and AST liver enzymes; or
wherein the human subject possesses liver enzyme elevations within 3 times the upper limit of normal, to assume close to normal liver histology; or
wherein should the human subject have elevated liver enzymes and still require treatment, then the human subject requires a DNA dose at least 50 mg per kilogram of liver weight to compensate for distorted pathology; or
wherein the human subject has fulminant hepatitis induced by Wilson's Disease and is administered via an endoscopic procedure with an elevated non-viral DNA dose of at least 50 mg per kg of liver weight to compensate for distorted pathology; or
wherein the efficacy of the non-viral DNA vector is monitored with urinary copper, serum copper, ceruloplasmin, and/or biopsy-gained hepatic copper measurements; or
wherein the subject is preferentially selected among patients with Wilson's disease who do not tolerate current copper chelation medications, or alternatively have neurological disease with any treatment response, or alternatively have antibodies against AAV capsids; or
wherein after the administration step, the patient may start or continue taking copper chelation medications and zinc for a period of at least 1-month, at least 2 months, at least 3 months, or at least 6 months to help accelerate the de-coppering process from the body, before cessation of pharmacologic therapy; or
wherein the combination therapy will prevent hepatocyte turnover from copper-induced death before the genetic treatment has time to take effect; or
wherein the non-viral DNA vector is maintained either as an episome without integration, or alternatively is facilitated with integration; or
wherein human subject is redosed with episomal vectors configured for redosing at least once every one year, at least every two years, at least every three years, or at least every five years; or
wherein the need for redosing is determined by assessing the elevation in the ALT and AST liver enzymes, such that enzymes fall outside the upper limit of normal; or
wherein the non-viral DNA is integrated in the genome of the human subject, optionally via a transposase, a large serine recombinase, or a CRISPR-directed homologous recombination; or
wherein the non-viral DNA vector yields a selective proliferative advantage of liver cells harboring the DNA vector in a Wilson's Disease individuals' liver over time increasing the amount of those liver cells positive for the vector-derived ATP7B, optionally wherein no other exogenous agents or chemicals or partial hepatectomy are needed to induce this proliferative advantage, optionally wherein the proliferative advantage can be slowed through the administration of copper chelation and zinc as desired; or
wherein hepatocytes and cholangiocytes of the liver are targeted for expression with ATP7B; or
wherein the non-viral DNA vector is integrated into host hepatocytes or cholangiocytes for replication with mitosis to provide stability due to the proliferative advantage and/or turnover of un-transfected hepatocytes, optionally via a transposon system; or
wherein the transposon system is a piggyBac transposon, a hyperactive piggyBac transposon, or a Sleeping Beauty Transposon; or
wherein the non-viral DNA vector alternatively harbors sequence elements to enable episomal replication for replication with mitosis; or
wherein the episomal replication sequence is a scaffold/matrix attachment region sequence; or
wherein the administered non-viral vector is a double-stranded circular or linear DNA; or
wherein the non-viral DNA vector includes a promoter, a 5′ UTR, a human ATP7B coding sequence, a 3′ UTR, an enhancer and polyadenylation sequence; or
wherein the promoter is selected from the group consisting of a hepatocyte-specific promoter, consisting of alpha-1 antitrypsin, human thyroxine binding globulin, hemopexin, albumin, LP1, P3, and mouse transthyretin promoters.

3-29. (canceled)

30. The method of claim 1, wherein the promoter is selected from the group consisting of a cholangiocyte-specific promoter, consisting of cytokeratin-17, cytokeratin-19, cyclooxygenase-2 (COX-2), midkine (MK), mucin-1 (MUC1), and osteopontin.

31. The method of claim 1 wherein the promoter is a tandem of a hepatocyte- and cholangiocyte-specific promoter, with the cholangiocyte 5′ in order to the hepatocyte promoter, thereby allowing ATP7B expression in both hepatocytes and cholangiocytes.

32. The method of claim 1, wherein a promoter is selected that has expression in both hepatocytes and cholangiocytes, such as cytokeratin-18 promoter, or the alpha-1 antitrypsin promoter.

33. The method of claim 1, wherein the enhancer element is added to the promoter, consisting of a liver-specific enhancer such as human apolipoprotein hepatic control region, human albumin enhancer, human ApoE enhancer, or a viral enhancer such as HBV enhancer I, HBV enhancer II to drive more potent expression.

34. The method of claim 1, wherein non-viral DNA vector optionally contains at least one intron selected from SV40 intron, Minute Virus of Mice (MVM) intron, and human growth hormone (HGH) intron, preferably in the 5′ UTR to enhance ATP7B expression.

35. The method of claim 1, wherein the coding sequence for ATP7B is codon-optimized for human hepatocyte expression, such that the expression level is at least 2-fold higher than the wild-type ATP7B sequence.

36. The method of claim 1 wherein the 3′ UTR is selected among human beta-globin UTR, human alpha-globin UTR, or a doublet of those sequences for added stability.

37. The method of claim 1, wherein the non-viral DNA vector does not induce overexpression toxicity of ATP7B in large animals compared to rodents when utilizing constitutively active liver-specific promoters due to differences in delivery efficiency, such that no native regulatory elements are required.

38. The method of claim 1, wherein expression of ATP7B is constitutive and thus can suppress the endogenous mutant ATP7B, which is regulated by copper levels, thereby enhancing the therapeutic effect by alleviating the malfunctioning form inside the cell.

39. The method of claim 1, wherein the native ATP7B promoter, or other promoter controlled by metal-responsive elements are preferably not used in the viral vector to avoid promoter competition with the endogenous mutant proteins.

40. The method of claim 1, wherein the subject is further administered with copper supplements if vector expression from ATP7B be too significant and cause copper deficiency

41. The method of claim 1, wherein the native ATP7B mutant's expression is knocked down with shRNA that is also encoded on the non-viral vector, such that the new delivered wildtype ATP7B avoids mispairing with the mutant receptor inside the cell.

42. The method of claim 1, wherein hydrodynamic injection of DNA occurs alternatively through the hepatic vein, or hepatic artery to mediate delivery into liver cells.

43. The method of claim 1, wherein hepatocytes expressing ATP7B after non-viral delivery will expand at least 2-fold, 3-fold, 4-fold, or 5-fold or more after at least 2 months post-gene delivery compared to their original number post-injection.

44. The method of claim 1, wherein the ATP7B protein coding sequence can tolerate an N-terminal or C-terminal additions for protein identification without affecting protein function.

45. The method of claim 1, wherein the subject is a canine subject who possesses a pathologic mutation in ATP7B and elevated hepatic copper levels causing silent or active hepatitis.

46. The method of claim 45, wherein the canine subject will receive wildtype canine ATP7B (NM_001025267.1) into the canine liver.

47. The method of claim 45, wherein the canine subject will preferentially receive integrative vector strategies to avoid the need for future redosing.


SEQUENCE LISTING (14 sequences)

SEQ ID NO: 1 - DNA, 1070 bp, Homo sapiens
  source 1..1070: mol_type = genomic DNA
acaaggaaggccatttgcccgcaaaatttagctacactggacgggcaagtacccctacagaagagaaaacgtctgtgagcccacacgaccggctgctcacctcaacaacttgcacaggcaccagctcctttcgccggccgccatcttccgccgacccccgaactcaggaaacgcttcactttccttttccctattggctcctgagaaagcaagccgtgctcgccccgcccccacgggccaattgtgcgttactattggttactggtagccgcttcccacggccttccagccaatagaatatgccgaggcgtagactagtgttcggcgtggcgcacacggctcccgcccccgtgggcgggacagcagtggggggttgggctgaggagggcgtggcctgtgattgacagccgtcgctccctccctcggccacctcccccactagaagcccccgcctgggcgcctgcgcccccgttcccggcccaaagcccgccgcccgttggaggccattggctggcctttgcgcacagcggatcgattttccaggtgcggagttcactcttgccgcggttgcttcctttgggacccacggcgtccggcagccaggcgcagagtccgaggagggggcagcgcagagcggacccgacgcggcgccgccgggcaccttccccgcaggcggtgggtgagccctgggagctgagtctgcggtccggctctgcgcagctcacctgccctcccgctcccgcacacgcgtgagatcccagtacagtgtcggagcgcaccagcgcgaggtggccgagaccgcggaggaggacaggcctccgccctgcggcgccggcacggcagaggacattgtggcactggcacggcagagaacactgtggcaccggcggggccggcagttccagggtgggcactcccagccacctggggagtgggcgagggtccgaggcccactctcccctcacgctctcatccccgtgcccccaggtcgggaggacggcggcgcgcaactttgaatcatccgtgtgaagagggctgcggcttccccggtcccaaatgaaggggcggtt

SEQ ID NO: 2 - DNA, 987 bp, Homo sapiens
  source 1..987: mol_type = genomic DNA
cacgaccggctgctcacctcaacaacttgcacaggcaccagctcctttcgccggccgccatcttccgccgacccccgaactcaggaaacgcttcactttccttttccctattggctcctgagaaagcaagccgtgctcgccccgcccccacgggccaattgtgcgttactattggttactggtagccgcttcccacggccttccagccaatagaatatgccgaggcgtagactagtgttcggcgtggcgcacacggctcccgcccccgtgggcgggacagcagtggggggttgggctgaggagggcgtggcctgtgattgacagccgtcgctccctccctcggccacctcccccactagaagcccccgcctgggcgcctgcgcccccgttcccggcccaaagcccgccgcccgttggaggccattggctggcctttgcgcacagcggatcgattttccaggtgcggagttcactcttgccgcggttgcttcctttgggacccacggcgtccggcagccaggcgcagagtccgaggagggggcagcgcagagcggacccgacgcggcgccgccgggcaccttccccgcaggcggtgggtgagccctgggagctgagtctgcggtccggctctgcgcagctcacctgccctcccgctcccgcacacgcgtgagatcccagtacagtgtcggagcgcaccagcgcgaggtggccgagaccgcggaggaggacaggcctccgccctgcggcgccggcacggcagaggacattgtggcactggcacggcagagaacactgtggcaccggcggggccggcagttccagggtgggcactcccagccacctggggagtgggcgagggtccgaggcccactctcccctcacgctctcatccccgtgcccccaggtcgggaggacggcggcgcgcaactttgaatcatccgtgtgaagagggctgcggcttccccggtcccaaatgaaggggcggtt

SEQ ID NO: 3 - DNA, 160 bp, Homo sapiens
  source 1..160: mol_type = genomic DNA
cacgaccggctgctcacctcaacaacttgcacaggcaccagctcctttcgccggccgccatcttccgccgacccccgaactcaggaaacgcttcactttccttttccctattggctcctgagaaagcaagccgtgctcgccccgcccccacgggccaatt

SEQ ID NO: 4 - DNA, 93 bp, Homo sapiens
  source 1..93: mol_type = genomic DNA
ctctaaggtaaatataaaatttttaagtgtataatgtgttaaactactgattctaattgtttctctcttttagattccaacctttggaactga

SEQ ID NO: 5 - protein, 1465 aa, Homo sapiens
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIKSKVAPLSLGPIDIERLQSTNPKRPLSSANQNFNNSETLGHQGSHVVTLQLRIDGMHCKSCVLNIEENIGQLLGVQSIQVSLENKTAQVKYDPSCTSPVALQRAIEALPPGNFKVSLPDGAEGSGTDHRSSSSHSPGSPPRNQVQGTCSTTLIAIAGMTCASCVHSIEGMISQLEGVQQISVSLAEGTATVLYNPSVISPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 6 - DNA, 4398 bp, Homo sapiens
  source 1..4398: mol_type = genomic DNA
atgcctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgcagaaggaaaggcagcctcctggccctcaaggtccttgcctgcccaggaggctgtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcaagagcaaagtggctcccttaagcctgggaccaattgatattgagcggttacaaagcactaacccaaagagacctttatcttctgctaaccagaattttaataattctgagaccttggggcaccaaggaagccatgtggtcaccctccaactgagaatagatggaatgcattgtaagtcttgcgtcttgaatattgaagaaaatattggccagctcctaggggttcaaagtattcaagtgtccttggagaacaaaactgcccaagtaaagtatgacccttcttgtaccagcccagtggctctgcagagggctatcgaggcacttccacctgggaattttaaagtttctcttcctgatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatccctctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctgtgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctga

SEQ ID NO: 7 - protein, 1474 aa, Homo sapiens
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIKSKVAPLSLGPIDIERLQSTNPKRPLSSANQNFNNSETLGHQGSHVVTLQLRIDGMHCKSCVLNIEENIGQLLGVQSIQVSLENKTAQVKYDPSCTSPVALQRAIEALPPGNFKVSLPDGAEGSGTDHRSSSSHSPGSPPRNQVQGTCSTTLIAIAGMTCASCVHSIEGMISQLEGVQQISVSLAEGTATVLYNPAVISPEELRAAIEDMGFEASVVSESCSTNPLGNHSAGNSMVQTTDGTPTSLQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAAPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYITETSQVAPA

SEQ ID NO: 8 - DNA, 186 bp, Homo sapiens
  source 1..186: mol_type = genomic DNA
atgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatccgataaggatcgatccgggc

SEQ ID NO: 9 - DNA, 800 bp, Homo sapiens
  source 1..800: mol_type = genomic DNA
agtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggc

SEQ ID NO: 10 - DNA, 186 bp, Homo sapiens
  source 1..186: mol_type = genomic DNA
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 11 - DNA, 398 bp, Homo sapiens
  source 1..398: mol_type = genomic DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 12 - DNA, 740 bp, Homo sapiens
  source 1..740: mol_type = genomic DNA
gcctgtaatcccagcactttggacggcccaggcgggtggatcacttgaggtcaggagtttgagaccagcctggtcaacatggtgaaaccctgtctctactaaaaatacaaaaattagctgggcgtggtggcgcgtgcctgtaaccccagctactccggaggctgaggcaggagaatcgcttgaacccgggagatggaggctgcagtgggccgagatcacaccactgcactccagtctgggcgacagagactcgtctccaaaaaaacaaaaacaaaatcactgggtcaggggtgtgtaggaatatgaccccagagggactgtaattcccagtggtgtcaaactctgggttgatcttaaagggtgaggctcagaatgtggcttccagggacaggggtgtccaagtatgtccctgacgggggaaaggccagaacagggtctgcagagagagagtgggggagtgcgggtcggagcttctgcgcggaccggggcggggcacctctggagggcaggggcctctggtctctgggaggggagggaattgaccaatggggagagagcccatatttgctctcaggagcctgcaaattcctcagggctcagatatccgcccctgacaccattcctcccttcccccctccaccggccgcgggcataaaaggcgccaggtgagggcctcgccgctcctcccgcgaatcgcagcttctgagaccagggttgctccgtccgtgctccgcctcgcc

SEQ ID NO: 13 - DNA, 3311 bp, Homo sapiens
  source 1..3311: mol_type = genomic DNA
caataacagtaaaaggcagtacgtagcttgttgactccacatactttattataaaatactgcccaacttgacagttctggaatccagtgggggaatataaaggtgaaagcaggagagacccctctgactggaacctcttacctcccagaagccttgtatgcaaaaccagtgggcattcatttgtatgttattttgcatcccgtttgcctcccagccttcagcaggccccgaccctcccctggccagcttccaccctgactgccccctggctggctcccattgagcactgtggggctctccccaccattaggtgacagatcaggaacaatccaggctcaggctctttatctgtgctctgcctcccacctggcaggtccactggccaggcttttccagggtcccttctctcycaggtctgccctactatttgtcctcccctccccctcagctggtagctcgataagaatcaataggtccactccagagcaaagaacacagccaaatgtgtcataccaggccctgccagaaaaacgagctgctggagctgacaaacttgaaggccaaacacctaagggttccccccaacacttcattcagcagggatggtcattcagcttcagggggcaggcagcatgaaagcctccctacctccatccttctcacacagaggctggggagagcatcttggaggatgcagtcccctggggccaggcttctaatccagacagcccttacaaggggggacaggggaaggactggcttggagaaaagtcctagaaaagaggggaggggcactggccaccagggctgggtcgctgctatgatggtcctaggagtgcctgcctgtcctctcaggccccatgcgatgtaggacacattacttttatttatttatttatttatttattttgagtcagagtttcgctctggttgcccaggctggagcgcgacggcacgatcttggctcactgcaacctctgcctcctgggttcaagcgattctcctgcctcagcctcctgagtagctgggattacaggcacacactgtgcctggttaatttttgtatttttagtagagaaggggtgtcaccatgttggtcaggctggtctcaaatttttttttttttttttttttttgagacagagtcttgctctgttgtctaggctggagtgcagtggcatcgaactcttgacctcaagtgatccacccgcctcggcctcccaaagtgcttggattacaggcatgagccactgtgcccggcgatgtgggacacattatcatctctgtgagagatttttggtctcttttgtcaccgcccttctctcccagctcctagaactgggcctggctcacagtaggtgctgaatgcatactggttgaattgtaaatgctcaggatttgtttaattaaggatgcaggaaaggtgatataccggtgtgcagaagtcaggatgcattccctgtccaaatcacagtgttccactgaggcaaggcccttgggagtgaggtcgggagaggggagggtggtggagggggctcagagactgggtttgttttggggagtctgcacctatttgctgagtgaatgtatgtgtgtgtgcatttgagagcacacctctgtatgattcgggtgtgagtgtgtgtgaggaaacgtgggcaggcgaggagtgtttgggagccaggtgcagctggggtgtgagtgtgtaagcaagcagctatgaggctgggcattgcttctcctcctcttctccagctcccagcctttcttccccgggactcctggggctccaggatgcccccaagatcccctccacaagtggataatttgggctgcaggttaaggacagctagagggactcacaggccattccacccgcacaccaccagacccccaaatttcttttttcttttttttttttttttttttttgagacagagtctcactctgtcgccaggctgcagtggcgcgatctcggctcactgcaacctccgcctcccaggttcaagcgattccccttcctcagcctcccaagtagctgaractacaggcgtgcaccatcacgtccggctaattttttgtattttagtagagagggggtttcaccatgttggctaggatggtctcgatctcctgacctcgtgatccgcccacctaggcctcccaaagtgctgagattacaggcgtgagccactgcgcccggtcaagactcccaaatttcaaactcgccagcacctcctccacctgggggagaagagcataataacgtcatttcctgccctgaaagcagcctcgagggccaacaacacctgctgtccgtgtccatgcccggttggccaccccgtttctggggggtgagcggggcttggcagggctgcgcggagggcgcgggggtggggcccggggcggagcggcccggggcggagggcgcgggctccgagccgtccacctgtggctccggcttccgaagcggctccggggcgggggcggggcctcactctgcgatataactcgggtcgcgcggctcgcgcaggccgccaccgtcgtccgcaaagcctgagtcctgtcctttctctcacgcgtcaggtaaaggggtaggagggacctcaactccccagccttgtctgacccctcccaattattacactcctttgcctcttccgtcattcccataaccacccaaccctactccaccgggagggggttgggcatacctggatttccatccgcgcacctagccacagggtccctaagagcagcagctaggcatgggagggctctttcccaggagagagggggaaggggacagggttgagagctttacagaggaagtggacagcatggagggaggtaaggaaaggcctgtaaagaggaggagacactggctctggcggaatggggactattggagggttaagcggatgtggctaaggctgagtcatctaggagtaaacaagaggccttcctttgggaggagccaatccagggtgtagggggcccagagtgaccaggtgcactagggaaaaaatgccaggagagggccaggaagaggacttgttagtagcgactcacttctgggcaggcaggccagccagctagccagcctgctgaggcttcccaagaggggcagagtgctgggatctgggaatccaggaaaggagggaatggggtggggctagatgaaaagggataggtgtccagggagagcctctggctattcctgggaccaggaagttttcactaggatacataacactttttacacactcaccccacccatccctggctttctattcatggaacaacctctctct

SEQ ID NO: 14 - DNA, 5900 bp, Homo sapiens
  source 1..5900: mol_type = genomic DNA
ttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgtggaggggctagctcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccaccatgcctgaacaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcaggtggccaccagcacagtcaggatcttgggcatgacttgccagtcatgtgtgaagtccattgaggacaggatttccaatttgaaaggcatcatcagcatgaaggtttccctggaacaaggcagtgccactgtgaaatatgtgccatcggttgtgtgcctgcaacaggtttgccatcaaattggggacatgggcttcgaggccagcattgcagaaggaaaggcagcctcctggccctcaaggtccttgcctgcccaggaggctgtggtcaagctccgggtggagggcatgacctgccagtcctgtgtcagctccattgaaggcaaggtccggaaactgcaaggagtagtgagagtcaaagtctcactcagcaaccaagaggccgtcatcacttatcagccttatctcattcagcccgaagacctcagggaccatgtaaatgacatgggatttgaagctgccatcaagagcaaagtggctcccttaagcctgggaccaattgatattgagcggttacaaagcactaacccaaagagacctttatcttctgctaaccagaattttaataattctgagaccttggggcaccaaggaagccatgtggtcaccctccaactgagaatagatggaatgcattgtaagtcttgcgtcttgaatattgaagaaaatattggccagctcctaggggttcaaagtattcaagtctccttggagaacaaaactgcccaagtaaagtatgacccttcttgtaccagcccagtggctctgcagagggctatcgaggcacttccacctgggaattttaaagtttctcttcctgatggagccgaagggagtgggacagatcacaggtcttccagttctcattcccctggctccccaccgagaaaccaggtccagggcacatgcagtaccactctgattgccattgccggcatgacctgtgcatcctgtgtccattccattgaaggcatgatctcccaactggaaggggtgcagcaaatatcggtgtctttggccgaagggactgcaacagttctttataatcccgctgtaattagcccagaagaactcagagctgctatagaagacatgggatttgaggcttcagtcgtttctgaaagctgttctactaaccctcttggaaaccacagtgctgggaattccatggtgcaaactacagatggtacacctacatctctgcaggaagtggctccccacactgggaggctccctgcaaaccatgccccggacatcttggcaaagtccccacaatcaaccagagcagtggcaccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagaaatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagccccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatctgatgaagatctcatcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatccgataaggatcgatccgggctcgagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaa